(19) United States
(12) Patent Application Publication
(10) Pub. No.: US 2022/0257935 A1
(43) Pub. Date: Aug. 18, 2022

(54) SYSTEMS AND METHODS FOR NERVE FIBER CONDUCTION BLOCK

(71) Applicant: Duke University, Durham, NC (US)

(72) Inventors: Edgar Pena, Durham, NC (US); Nicole A. Pelot, Durham, NC (US); Warren Grill, Durham, NC (US)

(21) Appl. No.: 17/674,212

(22) Filed: Feb. 17, 2022

Related U.S. Application Data
(60) Provisional application No. 63/150,658, filed on Feb. 18, 2021.

(51) Int. Cl. A61N 1/06; A61N 1/05; A61B 5/279

(52) U.S. Cl. CPC A61N 1/06; A61B 5/279; A61N 1/0529

(57) ABSTRACT

The present disclosure provides systems and methods relating to neuromodulation. In particular, the present disclosure provides systems and methods for selective and/or unidirectional nerve fiber conduction block though the application of a hybrid waveform using a neuromodulation device. The systems and methods of neuromodulation disclosed herein facilitate the treatment of various diseases associated with pathological neural activity.

### RELATED APPLICATIONS

This application claims priority to and the benefit of U.S. Provisional Patent Application No. 63/150,658 filed Feb. 18, 2021, which is incorporated herein by reference in its entirety for all purposes.

### GOVERNMENT FUNDING

This invention was made with Government support under Federal Grant No. OT2 OD025340 awarded by National Institutes of Health. The Federal Government has certain rights to the invention.

### FIELD

The present disclosure provides systems and methods relating to neuromodulation. In particular, the present disclosure provides systems and methods for selective and/or unidirectional nerve fiber conduction block though the application of a hybrid waveform using a neuromodulation device. The systems and methods of neuromodulation disclosed herein facilitate the treatment of various diseases associated with pathological neural activity.

### BACKGROUND

Implanted neural stimulation devices for the treatment of disease are widespread and typically deliver electrical signals at tens to hundreds of hertz to evoke neural activity. Less widely used are kilohertz frequency (KHF) waveforms that can block conduction of neural activity. KHF signals produce persistent mean depolarization of the axonal membrane near the electrode contacts, causing sodium channel inactivation and local conduction block. Preclinical studies of KHF nerve block for a wide range of disorders including diabetes, heart failure, and bladder control reflect the potential of this emerging technology. However, the relationship between waveform parameters and the nerve fibers that are blocked is poorly understood and this limits the ability to block selectively targeted nerve fibers.

Although most studies of KHF block report that the minimum current amplitude to achieve block increases with signal frequency, some previous studies showed a non-monotonic effect of signal frequency on block threshold. For example, in experiments on rat vagus and sciatic nerves, using sinusoidal KHF signals, frequencies ≤30 kHz blocked faster conducting fibers at lower thresholds, while frequencies ≥50 kHz blocked more slowly conducting fibers at lower thresholds; this raises the important possibility of fiber-type selective block by choosing an appropriate signal frequency. However, these findings were not replicated in a subsequent studies in which both slow and fast conducting fibers of the rat vagus nerve exhibited monotonically increasing block thresholds with frequency, and the slow fibers had higher block thresholds at all frequencies. Non-monotonic frequency effects are unexpected because the passive properties of the axonal membrane attenuate high frequencies irrespective of fiber diameter or myelination, and this attenuation underlies the increase in block thresholds at higher frequencies. The non-monotonic thresholds in the previous studies may be due to unintended charge imbalances in the waveforms generated by the instrumentation, which modulated the threshold-frequency relationships; this explanation is consistent with computational modeling studies of charge-imbalanced asymmetric waveforms which also produced non-monotonic block thresholds. However, those modeling results did not clarify the relative roles of charge imbalance and waveform asymmetry in determining block thresholds, and the lack of experimental data limits the relevance to in vivo applications. In vivo data are particularly crucial given the potential of direct current (DC) to damage nerves, potentially limiting long-term use of this technique.

### SUMMARY

Embodiments of the present disclosure include a method for selective nerve fiber conduction block using a neuromodulation device. In accordance with these embodiments, the method includes applying a hybrid waveform comprising a kilohertz frequency (KHF) component and a direct current (DC) component to a target nerve fiber or set of nerve fibers such that the hybrid waveform achieves conduction block in the target nerve fiber or set of nerve fibers.

In some embodiments, the KHF component comprises a biphasic alternating current waveform. In some embodiments, the KHF component comprises a waveform with more than two phases.

In some embodiments, the DC component comprises a DC offset superimposed on the KHF component. In some embodiments, the DC component comprises unequal phase durations and/or unequal phase amplitudes in the KHF component.

In some embodiments, the hybrid waveform is repeated at a frequency of about 1 kHz to about 200 kHz.

In some embodiments, the hybrid waveform comprises a net charge imbalance per unit time. In some embodiments, the net charge imbalance is obtained by: (a) adjusting the amplitude of the DC offset superimposed on the KHF component; (b) adjusting the magnitude of the difference in the phase durations of the KHF component; (c) adjusting the magnitude of the difference in the amplitudes of the phases of the KHF component; and/or (d) adjusting the shapes of the phases of the KHF component; and any combinations of (a)-(d).

In some embodiments, the method further comprises adjusting polarity of the DC component.

In some embodiments, the hybrid waveform blocks conduction in the target nerve fiber or set of nerve fibers but does not block conduction in a reference nerve fiber or set of nerve fibers. In some embodiments, the target nerve fiber or set of nerve fibers comprises a diameter(s) that is smaller than the reference nerve fiber. In some embodiments, the reference nerve fiber comprises a diameter that is from about 0.5 μm to about 20.0 μm. In some embodiments, the target nerve fiber or set of nerve fibers comprises a diameter(s) from about 0.2 μm to about 19.5 μm. In some embodiments, the hybrid waveform comprises a repetition frequency of about 1 kHz to about 200 kHz. In some embodiments, the hybrid waveform comprises a charge imbalance obtained by: (a) adjusting unequally the amplitudes of the phases of the KHF component; (b) adjusting the magnitude of the difference in the phase duration of the KHF component; (c) adjusting the amplitude of the DC offset superimposed on the KHF component; and/or (d) adjusting the shapes of the phases of the KHF components; and any combinations of (a)-(d).

In some embodiments, the DC component comprises an anodal charge imbalance or a cathodal charge imbalance. In some embodiments, the DC component comprises an amplitude of greater than or equal to about 1 μA per milliamp of the KHF component per kilohertz of the KHF component. In some embodiments, the KHF component comprises an amplitude between 0.1 mA to 20 mA.

In some embodiments, the hybrid waveform blocks conduction in the target nerve fiber or set of nerve fibers but does not block conduction in a reference nerve fiber or set of nerve fibers. In some embodiments, the target nerve fiber or set of nerve fibers comprises a diameter(s) that is larger than the reference nerve fiber. In some embodiments, the reference nerve fiber comprises a diameter that is from about 0.2 μm to about 19.5 μm. In some embodiments, the target nerve fiber or set of nerve fibers comprises a diameter(s) from about 0.5 μm to about 20.0 μm. In some embodiments, the hybrid waveform comprises a repetition frequency of about 1 kHz to about 200 kHz. In some embodiments, the hybrid waveform comprises a charge imbalance obtained by: (a) adjusting unequally the amplitudes of the phases of the KHF component; (b) adjusting the magnitude of the difference in the phase duration of the KHF component; (c) adjusting the amplitude of the DC offset superimposed on the KHF component; and/or (d) adjusting the shapes of the phases of the KHF components; and any combinations of (a)-(d).

In some embodiments, the DC component comprises an anodal charge imbalance or a cathodal charge imbalance. In some embodiments, the DC component comprises an amplitude of 0 μA to 100 μA per milliamp of KHF component per kilohertz of the KHF component. In some embodiments, the KHF component comprises an amplitude of 0.1 mA to 20 mA.

In some embodiments, the hybrid waveform blocks conduction in a unidirectional manner. In some embodiments, the hybrid waveform comprises a repetition frequency of about 1 kHz to about 200 kHz. In some embodiments, the hybrid waveform comprises a charge imbalance obtained by: (a) adjusting unequally the amplitudes of the phases of the KHF component; (b) adjusting the magnitude of the difference in the phase duration of the KHF component; (c) adjusting the amplitude of the DC offset superimposed on the KHF component; and/or (d) adjusting the shapes of the phases of the KHF components; and any combinations of (a)-(d). In some embodiments, the DC component comprises an anodal charge imbalance or a cathodal charge imbalance. In some embodiments, the DC component comprises an amplitude of greater than or equal to about 1 μA per milliamp of the KHF component per kilohertz of the KHF component. In some embodiments, the KHF component comprises an amplitude between 0.1 mA to 20 mA.

Embodiments of the present disclosure also include a system for selective nerve fiber conduction block. In accordance with these embodiments, the system includes an electrode with one or more metal contacts sized and configured for implantation in proximity to neural tissue, and a pulse generator coupled to the electrode, the pulse generator including a power source comprising a battery and a microprocessor coupled to the battery. In some embodiments, the pulse generator is capable of applying to the electrode a hybrid waveform capable of achieving selective conduction block in a target nerve fiber or set of nerve fibers.

In some embodiments, the hybrid waveform comprises a KHF component comprising a biphasic alternating current waveform, and a DC component obtained by: (a) adjusting unequally the amplitudes of the phases of the KHF component; (b) adjusting the magnitude of the difference in the phase duration of the KHF component; (c) adjusting the amplitude of the DC offset superimposed on the KHF component; and/or (d) adjusting the shapes of the phases of the KHF components; and any combinations of (a)-(d).

In some embodiments, the hybrid waveform comprises a net charge imbalance per unit time. In some embodiments, the hybrid waveform blocks conduction in the target nerve fiber or set of nerve fibers but does not block conduction in a reference nerve fiber or set of nerve fibers.

Embodiments of the present disclosure also include a method for obtaining selective nerve fiber conduction block using any of the systems described herein; the method includes programming the pulse generator to output the hybrid waveform such that the hybrid waveform blocks neural conduction when delivered by the pulse generator.

Embodiments of the present disclosure also include a method for obtaining unidirectional nerve fiber conduction block using a neuromodulation device. In accordance with these embodiments, the method includes applying a hybrid waveform comprising a kilohertz frequency (KHF) component and a direct current (DC) component to a target nerve fiber or set of nerve fibers, such that the hybrid waveform achieves a conduction block in the target nerve fiber or set of nerve fibers in a unidirectional manner.

In some embodiments, the KHF component comprises a biphasic alternating current waveform. In some embodiments, the KHF component comprises a waveform with more than two phases. In some embodiments, the DC component comprises a DC offset superimposed on the KHF component. In some embodiments, the DC component comprises unequal phase durations or unequal amplitudes of phases in the KHF component. In some embodiments, the hybrid waveform is repeated at a frequency of about 1 kHz to about 200 kHz.

In some embodiments, the hybrid waveform comprises a charge imbalance obtained by: (a) adjusting unequally the amplitudes of the phases of the KHF component; (b) adjusting the magnitude of the difference in the phase duration of the KHF component; (c) adjusting the amplitude of the DC offset superimposed on the KHF component; and/or (d) adjusting the shapes of the phases of the KHF components; and any combinations of (a)-(d). In some embodiments, the DC component comprises an anodal charge imbalance or a cathodal charge imbalance. In some embodiments, the DC component comprises an amplitude of greater than or equal to about 1 μA per milliamp of the KHF component per kilohertz of the KHF component. In some embodiments, the KHF component comprises an amplitude between 0.1 mA to 20 mA. In some embodiments, the hybrid waveform blocks conduction in the target nerve fiber or set of nerve fibers but does not block conduction in a reference nerve fiber or set of nerve fibers.

Embodiments of the present disclosure also include a system for obtaining unidirectional nerve fiber conduction block. In accordance with these embodiments, the system includes an electrode with one or more metal contacts sized and configured for implantation in proximity to neural tissue, and a pulse generator coupled to the electrode, the pulse generator including a power source comprising a battery and a microprocessor coupled to the battery, such that the pulse generator is capable of applying to the electrode a hybrid waveform capable of achieving unidirectional conduction block in a target nerve fiber or set of nerve fibers.

In some embodiments, the hybrid waveform comprises a KHF component comprising a biphasic alternating current waveform, and a DC component obtained by: (a) adjusting unequally the amplitudes of the phases of the KHF component; (b) adjusting the magnitude of the difference in the phase duration of the KHF component; (c) adjusting the amplitude of the DC offset superimposed on the KHF component; and/or (d) adjusting the shapes of the phases of the KHF components; and any combinations of (a)-(d). In some embodiments, the hybrid waveform comprises a net charge imbalance per unit time. In some embodiments, the hybrid waveform blocks conduction in the target nerve fiber or set of nerve fibers but does not block conduction in a reference nerve fiber or set of nerve fibers.

Embodiments of the present disclosure also include a method for obtaining unidirectional nerve fiber conduction block using any of the systems described herein; the method includes programming the pulse generator to output the hybrid waveform such that the hybrid waveform blocks neural conduction in a unidirectional manner when delivered by the pulse generator.

### BRIEF DESCRIPTION OF THE DRAWINGS

FIGS. 1a-1e: Waveforms tested to independently analyze blocking effects of DC offset types, asymmetric charge imbalance, and asymmetry. KHF amplitude was defined as half of the peak-to-peak amplitude of each waveform. (a) Symmetric KHF waveform with zero net charge per unit time (Q=0). (b) Symmetric KHF waveforms with added DC offsets, where symmetry was defined as equal duration phases. Net charge per unit time (Q) was either negative (cathodal DC offset) or positive (anodal DC offset). (c) Types of DC offset added to symmetric KHF waveforms. DC offsets were either constant (i.e., independent of KHF parameters) (c1), amplitude-dependent (i.e., scaled with KHF amplitude only) (c2), or amplitude- and frequency-dependent (i.e., scaled with both KHF amplitude and frequency) (c3). (d) Asymmetric KHF rectangular waveforms were constructed from the symmetric waveform by defining unequal phase durations. Differences in phase durations (±2, 3, 4 μs) were independent of waveform frequency, such that the net charge per unit time scaled linearly with KHF amplitude and with frequency, analogous to the DC offset type illustrated in (c3). (e) Asymmetric waveforms with a compensatory DC offset that produced zero net charge per unit time.

FIGS. 2a-2b: Finite element model of rat tibial nerve with bipolar cuff electrode (a), and analogous in vivo experimental setup targeting rat tibial nerve (b). The “p” and “d” labels indicate the proximal and distal contacts, respectively. In the computational model in panel (a), test pulses were evoked near the proximal end and the transmembrane potential was recorded near the distal end of each axon modeled within the endoneurium. In the in vivo setup in panel (b), the cuff electrodes were placed on the sciatic nerve; the common peroneal and sural branches were transected (red X's), as well as the branches innervating the hamstring (not shown), and signals were transmitted to the gastrocnemius via the tibial branch.

FIGS. 3a-3f: Frequency effects on block thresholds in computational models of symmetric KHF rectangular waves with different types of DC offsets. Polarities apply to the proximal contact of the bipolar cuff (FIG. 2) and the signs of the DC offsets are for the current on the proximal contact. Model axons were myelinated and had a 5.7 μm fiber diameter. KHF amplitude indicates half of the peak-to-peak amplitude of the KHF waveform. (a & b) Block thresholds due to constant DC offset for cathodal (a) and anodal (b) polarities. The data for the four (a) and three (b) highest levels of DC are overlaid at zero threshold. Thresholds at 1,320 μA are not shown for cathodal DC because this amplitude produced only DC excitation. The black dotted line on the anodal DC plot shows the −186 μA data from the cathodal DC plot. (c & d) Block thresholds of KHF waveforms with cathodal (c) and anodal (d) DC offsets that scale with KHF amplitude. The black dotted line on the anodal DC plot shows the −200 μA per mA KHF data from the cathodal DC plot. (e & f) Block thresholds for KHF waveforms with cathodal (e) and anodal (f) DC offsets that scale with KHF amplitude and frequency. The black dotted line on the anodal DC plot shows the −4 μA per mA KHF per 1 kHz data from the cathodal DC plot.

FIGS. 4a-4b: Frequency effects on block thresholds during in vivo rat tibial nerve experiments. Symmetric KHF rectangular waves were offset by cathodal (a) or anodal (b) DC that scaled with KHF amplitude and frequency. Plots show mean and standard error of the mean of block thresholds across three to seven nerves. The black dotted line on the anodal DC offset plot shows the −4 μA per mA KHF per 1 kHz data from the cathodal DC offsets plot. See FIG. 9 for individual nerve data points.

FIGS. 5a-5b: Frequency effects of asymmetric waveforms in computational models (a) and in vivo experiments (b). Waveforms were either charge-balanced with asymmetric phases plus compensatory DC offsets to cancel out imbalances (top row; FIG. 1e) or charge-imbalanced with asymmetric phases (middle and bottom rows; FIG. 1d). KHF amplitude was half of the peak-to-peak amplitude of the KHF waveform for all waveforms. The amount of asymmetry is shown as the difference in duration between the first ((φ₁) and second ((φ₂) phases of the biphasic KHF waveforms. The black dotted line in each charge-imbalanced waveform plot shows the corresponding ±4 μA per mA KHF per 1 kHz line in silico data of FIG. 3 (computational model) or FIG. 4 (in vivo), which produced the same net charge per unit time as asymmetric charge-imbalanced waveforms with ±4 μs phase difference. In charge-balanced asymmetric waveforms, only negative phase differences are shown, as the sign of asymmetry had no effect on threshold-frequency relationships. The 0 μs phase difference (cyan) lines for in vivo data are from the same data as in FIG. 4. See FIG. 10 for individual nerve data points.

FIGS. 6a-6c: Computational models of block thresholds across frequencies for KHF and DC offset components separately in a 5.7 μm diameter fiber. (a) Original waveforms consisted of KHF symmetric rectangular waves with added DC offset that scaled with KHF amplitude and frequency. Digital high pass or low pass filters preserved only the KHF or DC components of the original signal, respectively. DC offsets were either cathodal (b) or anodal (c) at ±4 μA DC per mA pre-filtered KHF amplitude per 1 kHz. (b & c) KHF amplitude of Original waveform (y-axis) required for block with Original waveform (orange), KHF component only (cyan), or DC component only (purple). Threshold curves for original waveforms in (b) and (c) were identical to the corresponding ±4 μA DC per mA KHF amplitude per 1 kHz curves in FIGS. 3e-3f Threshold curves for KHF components in (b) and (c) were identical to the zero DC offset curves in all panels of FIG. 3. The black dotted line in the ‘DC Component’ panel of (c) shows the threshold curve from the cathodal DC component (b) for comparison.

FIGS. 7a-7c: KHF block across modeled axons of multiple fiber diameters without (a) and with (b, c) amplitude- and frequency-dependent DC offsets. Each model axon was placed at the center of the rat tibial nerve FEM, and all axon lengths were 100 mm.

FIG. 8: Representative examples of KHF amplitude and frequency effects on transmission, excitation, and block across a range of DC offset types for symmetric KHF waveforms from 10 to 100 kHz in computational models of 5.7 μm diameter myelinated fibers. Heatmaps show the number of action potentials that occurred between t=100 and 250 ms at all KHF amplitudes, frequencies, and polarities across a representative subset of DC offset levels from FIG. 3. Action potential counts were binned and color-coded (colorbar). The type, amount, and polarity of DC offsets are labeled above each plot. The signs of the DC offsets denote the polarity delivered to the proximal contact (FIG. 2). DC offset types are labeled above each group of plots. KHF amplitudes were sampled from 0.05 to 5 mA in 6% increments. Gray transmission dots indicate the presence of exactly three action potentials spaced apart in time by 50 ms, corresponding to the number and timing of test pulses between 100 to 250 ms. KHF amplitude was half of the peak-to-peak KHF waveform amplitude. Magenta lines show block threshold curves from corresponding panels in FIG. 3.

FIG. 9: Individual data across all seven nerves in symmetric waveforms for cathodal (top) and anodal (bottom) DC offsets.

FIG. 10: Individual data across all seven nerves in asymmetric waveforms for longer cathodal phase charge-imbalanced (top), longer anodal phase charge-imbalanced (middle), and charge-balanced asymmetric.

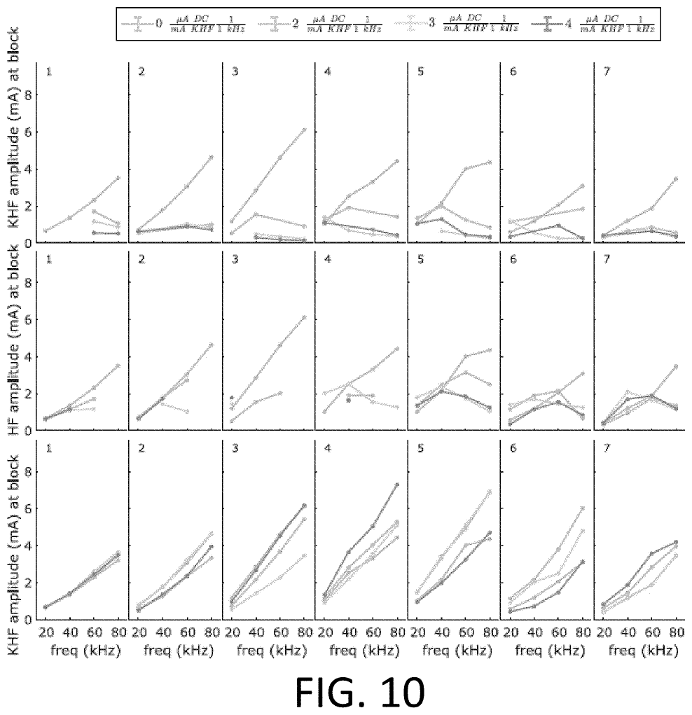

FIG. 11: Oscilloscope recordings (Tektronix tbs1032b) of kilohertz signals generated using the stimulator and load size reported in (Joseph and Butera 2009) (A-M Systems 2200, 30 k Ω resistive load) at two different amplitudes (1 mA & 1.25 mA) and two different frequencies (5 kHz & 50 kHz). DC offsets at 5 kHz were small (˜−13 μA DC per actual mA of KHF at 1 mA & 1.25 mA intended KHF), but DC offsets at 50 kHz were large (−164 and ˜−274 μA DC per actual mA of KHF at 1 mA & 1.25 mA intended KHF). The change in DC offsets per intended mA KHF from 5 kHz to 50 kHz was comparable to the DC offsets showed to be important for non-monotonic thresholds (˜−3.3 and ˜−5.8 μA DC per actual mA KHF per kHz). The stimulator was calibrated by adjusting DC offset screw to output <2 μA when the input was 0 V. Plots show average of four recorded cycles. DC offset was estimated from area under the curve of the average of the four recorded cycles using trapz in MATLAB R2018a.

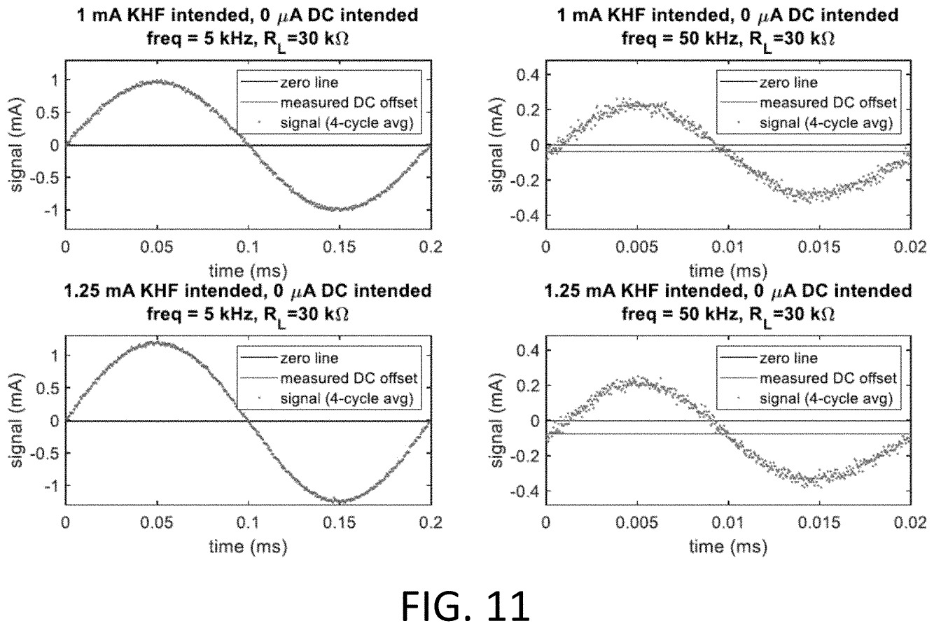

### DETAILED DESCRIPTION

Reversible block of nerve conduction using kilohertz frequency electrical signals has substantial potential for treatment of disease. However, the ability to block nerve fibers selectively is limited by poor understanding of the relationship between waveform parameters and the nerve fibers that are blocked. Previous in vivo studies reported non-monotonic relationships between block signal frequency and block threshold, suggesting the potential for fiber-selective block. However, the mechanisms of non-monotonic block thresholds were unclear, and these findings were not replicated in subsequent in vivo studies.

As described further herein, a comprehensive study was conducted to quantify the effects of charge imbalance, frequency, and asymmetry of KHF signals on block thresholds using computational models and in vivo experiments. The interactions between the KHF and DC contributions to conduction block were evaluated to investigate how frequency-dependent thresholds emerge from waveform characteristics. The results provided herein demonstrate that amplitude- and frequency-dependent charge imbalance resulted in non-monotonic block thresholds across frequencies, such that block was generated by the KHF component at low frequencies and by the DC component at high frequencies. The interactions between KHF and DC effects resulted in instances of block that were selective for smaller diameter model nerve fibers, and these interactions produced complex, polarity-dependent effects on block, transmission, and excitation across frequencies and KHF amplitudes. The data provided in the present disclosure provide the first experimental evidence of non-monotonic effects of frequency with charge-imbalanced waveforms, harmonize previous contradictory findings, and clarify the mechanisms of interaction between KHF and DC that can be leveraged for fiber-selective block.

As described further herein, the relationship between block threshold and block signal frequency can be controlled through manipulating the charge-imbalance of biphasic waveforms, whether through phase asymmetry or other charge-imbalanced KHF signals. Such methods, including asymmetric charge-imbalance waveforms, can be combined with slow charge recovery to eliminate net DC over time (see, e.g., Eggers T, Kilgore J, Green D, Vrabec T, Kilgore K, Bhadra N (2021) Combining direct current and kilohertz frequency alternating current to mitigate onset activity during electrical nerve block. J Neural Eng 18(4): 046010.)

Section headings as used in this section and the entire disclosure herein are merely for organizational purposes and are not intended to be limiting.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated. Recitation of ranges of values herein are merely intended to serve as a shorthand method of referring individually to each separate value falling within the range, unless otherwise-Indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

“Subject” and “patient” as used herein interchangeably refers to any vertebrate, including, but not limited to, a mammal (e.g., cow, pig, camel, llama, horse, goat, rabbit, sheep, hamsters, guinea pig, cat, dog, rat, and mouse, a non-human primate (e.g., a monkey, such as a cynomolgus or rhesus monkey, chimpanzee, etc.) and a human). In some embodiments, the subject may be a human or a non-human. In one embodiment, the subject is a human. The subject or patient may be undergoing various forms of treatment.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a treatment to a subject that is not at the time of administration afflicted with the disease. “Preventing” also refers to preventing the recurrence of a disease or of one or more symptoms associated with such disease.

“Therapy” and/or “therapy regimen” generally refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, neurobiology, microbiology, genetics, electrical stimulation, neural stimulation, neural modulation, and neural prosthesis described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### 2. NERVE FIBER CONDUCTION BLOCK

Reversible block of nerve activity using KHF electrical signals has potential applications across a wide range of diseases with pathophysiological neural activity. Reported non-monotonic relationships between block amplitude and signal frequency provide an exciting possibility to develop fiber-selective nerve block approaches, but these findings had to be reconciled with conflicting experimental evidence. Using high-fidelity computational models and in vivo experiments, the effects of KHF signals with a range of charge imbalances on KHF nerve block were quantified to clarify the mechanisms of non-monotonic threshold-frequency relationships. Block thresholds could indeed change non-monotonically with frequency, and non-monotonicity could result in smaller fibers being blocked at lower thresholds than larger fibers. These non-monotonic effects were due to amplitude- and frequency-dependent charge imbalances and not to waveform asymmetry.

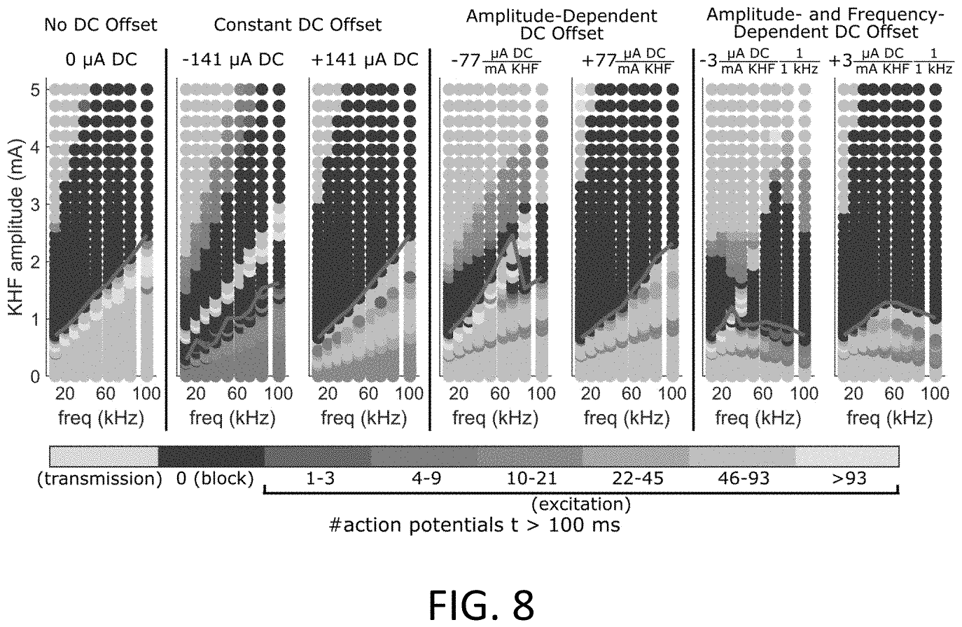

The effects of DC offset on KHF responses were complex and polarity-dependent. Polarity effects were particularly unexpected given the use of a geometrically symmetric bipolar cuff electrode. Nevertheless, the mechanism of these effects can be readily understood in terms of constructive or destructive interactions between depolarization resulting from the KHF and polarization by the DC anodal or cathodal offsets. The distal contact is particularly important to this understanding, as block can only be detected at the distal end of the axon if the distal contact blocks or if the proximal contact blocks in the absence of excitation at the distal contact. Low-amplitude DC anodal offsets at the proximal contact decreased KHF block thresholds because both the cathodal DC and the KHF signal at the distal contact drove membrane depolarization; low-amplitude cathodal DC at the proximal contact had the opposite effect because anodal DC at the distal contact counteracted KHF depolarization. Higher-amplitude DC of either polarity reduced block thresholds compared to pure KHF because, in those cases, block was primarily due to DC. However, anodal DC at the proximal contact had a weaker effect because the proximal anode caused sodium channel de-inactivation, which augmented incoming action potentials and enabled them to propagate through the distal cathode that would otherwise block. This phenomenon underlies the regions of transmission that emerged between excitation and block (e.g., FIG. 8, +141 μA DC), resulting in block of action potentials coming from one direction and transmission of action potentials coming from the opposite direction, and thus presenting the interesting possibility of unidirectional block with bipolar cuffs. Meanwhile, changes in KHF amplitude needed for re-excitation occurred because virtual DC cathodes (or virtual DC anodes) at the distal contact strengthened (or weakened) the depolarization at the virtual cathodes of the KHF signal, which are the source of KHF re-excitation. The observed polarity effects on block thresholds were consistent with in vivo DC block measurements from a previous study that used both monopolar and bipolar cuffs and with prior modeling of monopolar electrodes.

The data provided in the present disclosure used realistic preclinical computational models, which were validated with in vivo experiments. Further, the use of DC offsets, asymmetric waveforms, and asymmetric charge-balanced waveforms revealed that asymmetry was neither necessary nor sufficient for non-monotonic block thresholds across frequencies, but rather that charge imbalances that scale with KHF amplitude and frequency are required to cause non-monotonicity. Indeed, asymmetry in the absence of charge imbalance caused monotonic frequency effects with the same thresholds as for charge-balanced symmetric waveforms. The results of the present disclosure clarify that non-monotonic frequency effects represent a transition from KHF block to DC block. This transition exhibited complex characteristics beyond block threshold effects, such as the shifting, broadening, and even splitting of excitation regions (FIG. 8). These results are relevant to approaches seeking to implement DC offsets into KHF waveforms, as the alteration of excitation and block regions can reduce the available block window, making it harder to achieve and maintain nerve block.

The computational models of the present disclosure indicated that KHF waveforms with amplitude- and frequency-dependent charge imbalances enabled block of smaller fibers with lower amplitudes than larger fibers. In the light of advances in electrode materials that permit safe long-term DC nerve block, these results demonstrate that controlled DC offsets are a feasible approach for fiber-selective conduction block through tuning the KHF frequency and relative amount of DC offsets. Therefore, the findings of the present disclosure establish the utility of frequency for fiber-selective block, while elucidating the mechanism of action (e.g., DC offsets mixed with KHF), and indicate that block threshold changed non-monotonically with frequency when DC offsets scaled with KHF amplitude and frequency.

In accordance with the above, embodiments of the present disclosure include a method for selective and/or unidirectional nerve fiber conduction block using a neuromodulation device. In some embodiments, the method includes applying a hybrid waveform comprising a kilohertz frequency (KHF) component and a direct current (DC) component to a target nerve fiber or set of nerve fibers such that hybrid waveform achieves conduction block in the target nerve fiber or set of nerve fibers.

In some embodiments, the KHF component of the hybrid waveform comprises a biphasic alternating current waveform. In some embodiments, the KHF component of the hybrid waveform comprises a waveform with more than two phases. Additionally/alternatively, in some embodiments, the DC component of the hybrid waveform comprises a DC offset superimposed on the KHF component. In some embodiments, the DC component of the hybrid waveform comprises unequal phase durations and/or unequal phase amplitudes in the KHF component.

In some embodiments, the method for selective nerve fiber conduction includes applying the hybrid waveform at a repetition frequency of about 1 kHz to about 200 kHz. In some embodiments, the hybrid waveform is repeated at a frequency of about 1 kHz to about 175 kHz. In some embodiments, the hybrid waveform is repeated at a frequency of about 1 kHz to about 150 kHz. In some embodiments, the hybrid waveform is repeated at a frequency of about 1 kHz to about 125 kHz. In some embodiments, the hybrid waveform is repeated at a frequency of about 1 kHz to about 100 kHz. In some embodiments, the hybrid waveform is repeated at a frequency of about 1 kHz to about 75 kHz. In some embodiments, the hybrid waveform is repeated at a frequency of about 1 kHz to about 50 kHz. In some embodiments, the hybrid waveform is repeated at a frequency of about 25 kHz to about 150 kHz. In some embodiments, the hybrid waveform is repeated at a frequency of about 50 kHz to about 150 kHz. In some embodiments, the hybrid waveform is repeated at a frequency of about 75 kHz to about 150 kHz. In some embodiments, the hybrid waveform is repeated at a frequency of about 100 kHz to about 150 kHz. In some embodiments, the hybrid waveform is repeated at a frequency of about 125 kHz to about 150 kHz. In some embodiments, the hybrid waveform is repeated at a frequency of about 25 kHz to about 125 kHz. In some embodiments, the hybrid waveform is repeated at a frequency of about 50 kHz to about 100 kHz. In some embodiments, the hybrid waveform is repeated at a frequency of about 25 kHz to about 75 kHz. In some embodiments, the hybrid waveform is repeated at a frequency of about 50 kHz to about 125 kHz. In some embodiments, the hybrid waveform is repeated at a frequency of about 50 kHz to about 100 kHz. In some embodiments, the hybrid waveform is repeated at a frequency of about 75 kHz to about 125 kHz.

In some embodiments, the method for selective nerve fiber conduction block includes applying a hybrid waveform that comprises a net charge imbalance per unit time. In some embodiments, the net charge imbalance is obtained by adjusting the amplitude of the DC offset superimposed on the KHF component. In some embodiments, the net charge imbalance is obtained by adjusting the magnitude of the difference in the phase durations of the KHF component. In some embodiments, the net charge imbalance is obtained by adjusting the magnitude of the difference in the amplitudes of the phases of the KHF component. In some embodiments, the net charge imbalance is obtained by adjusting the shapes of the phases of the KHF component. In some embodiments, the net charge imbalance is obtained by any combinations of adjusting the amplitude of the DC offset superimposed on the KHF component, adjusting the magnitude of the difference in the phase durations of the KHF component, adjusting the magnitude of the difference in the amplitudes of the phases of the KHF component, and/or adjusting the shapes of the phases of the KHF component.

In some embodiments, the method for selective nerve fiber conduction block further includes adjusting polarity of the DC component. In some embodiments, adjusting the polarity of the DC component includes reversing the polarity of the DC component such that the direction of the block is reversed (e.g., unidirectional conduction block). In some embodiments, adjusting the polarity of the DC component includes using one or more electrical contacts (e.g., electrodes) with respect to the target nerve fiber or set of nerve fibers. In some embodiments, adjusting the polarity of the DC component includes using two or more electrical contacts (e.g., electrodes) with respect to the target nerve fiber or set of nerve fibers.

In some embodiments, the hybrid waveform blocks conduction in the target nerve fiber or set of nerve fibers but does not block conduction in a reference nerve fiber. In some embodiments, the target nerve fiber or set of nerve fibers comprises a diameter(s) that is smaller than the reference nerve fiber. In some embodiments, the reference nerve fiber comprises a diameter that is from about 0.5 μm to about 20.0 μm. In some embodiments, the reference nerve fiber comprises a diameter that is from about 1.0 μm to about 20.0 μm. In some embodiments, the reference nerve fiber comprises a diameter that is from about 1.5 μm to about 20.0 μm. In some embodiments, the reference nerve fiber comprises a diameter that is from about 2.0 μm to about 20.0 μm. In some embodiments, the reference nerve fiber comprises a diameter that is from about 2.5 μm to about 20.0 μm. In some embodiments, the reference nerve fiber comprises a diameter that is from about 3.0 μm to about 20.0 μm. In some embodiments, the reference nerve fiber comprises a diameter that is from about 3.5 μm to about 20.0 μm. In some embodiments, the reference nerve fiber comprises a diameter that is from about 4.0 μm to about 20.0 μm. In some embodiments, the reference nerve fiber comprises a diameter that is from about 4.5 μm to about 20.0 μm. In some embodiments, the reference nerve fiber comprises a diameter that is from about 5.0 μm to about 20.0 μm. In some embodiments, the reference nerve fiber comprises a diameter that is from about 5.5 μm to about 20.0 μm. In some embodiments, the reference nerve fiber comprises a diameter that is from about 6.0 μm to about 20.0 μm. In some embodiments, the reference nerve fiber comprises a diameter that is from about 6.5 μm to about 20.0 μm. In some embodiments, the reference nerve fiber comprises a diameter that is from about 7.0 μm to about 20.0 μm. In some embodiments, the reference nerve fiber comprises a diameter that is from about 7.5 μm to about 20.0 μm. In some embodiments, the reference nerve fiber comprises a diameter that is from about 8.0 μm to about 20.0 μm. In some embodiments, the reference nerve fiber comprises a diameter that is from about 8.5 μm to about 20.0 μm. In some embodiments, the reference nerve fiber comprises a diameter that is from about 9.0 μm to about 20.0 μm. In some embodiments, the reference nerve fiber comprises a diameter that is from about 9.5 μm to about 20.0 μm. In some embodiments, the reference nerve fiber comprises a diameter that is from about 10.0 μm to about 20.0 μm. In some embodiments, the reference nerve fiber comprises a diameter that is from about 10.5 μm to about 20.0 μm. In some embodiments, the reference nerve fiber comprises a diameter that is from about 11.0 μm to about 20.0 μm. In some embodiments, the reference nerve fiber comprises a diameter that is from about 11.5 μm to about 20.0 μm. In some embodiments, the reference nerve fiber comprises a diameter that is from about 12.0 μm to about 20.0 μm. In some embodiments, the reference nerve fiber comprises a diameter that is from about 12.5 μm to about 20.0 μm. In some embodiments, the reference nerve fiber comprises a diameter that is from about 13.0 μm to about 20.0 μm. In some embodiments, the reference nerve fiber comprises a diameter that is from about 13.5 μm to about 20.0 μm. In some embodiments, the reference nerve fiber comprises a diameter that is from about 14.0 μm to about 20.0 μm. In some embodiments, the reference nerve fiber comprises a diameter that is from about 14.5 μm to about 20.0 μm. In some embodiments, the reference nerve fiber comprises a diameter that is from about 15.0 μm to about 20.0 μm. In some embodiments, the reference nerve fiber comprises a diameter that is from about 15.5 μm to about 20.0 μm. In some embodiments, the reference nerve fiber comprises a diameter that is from about 16.0 μm to about 20.0 μm. In some embodiments, the reference nerve fiber comprises a diameter that is from about 16.5 μm to about 20.0 μm. In some embodiments, the reference nerve fiber comprises a diameter that is from about 17.0 μm to about 20.0 μm. In some embodiments, the reference nerve fiber comprises a diameter that is from about 17.5 μm to about 20.0 μm. In some embodiments, the reference nerve fiber comprises a diameter that is from about 18.0 μm to about 20.0 μm. In some embodiments, the reference nerve fiber comprises a diameter that is from about 18.5 μm to about 20.0 μm. In some embodiments, the reference nerve fiber comprises a diameter that is from about 19.0 μm to about 20.0 μm. In some embodiments, the reference nerve fiber comprises a diameter that is from about 19.5 μm to about 20.0 μm. In some embodiments, the reference nerve fiber comprises a diameter that is about 20.0 μm.

In accordance with the above embodiments, the target nerve fiber or set of nerve fibers is smaller than the reference nerve fiber and comprises a diameter(s) from about 0.2 μm to about 19.5 μm. In some embodiments, the target nerve fiber or set of nerve fibers comprises a diameter(s) that is from about 0.2 μm to about 19.0 μm. In some embodiments, the target nerve fiber or set of nerve fibers comprises a diameter(s) that is from about 0.2 μm to about 18.5 μm. In some embodiments, the target nerve fiber or set of nerve fibers comprises a diameter(s) that is from about 0.2 μm to about 18.0 μm. In some embodiments, the target nerve fiber or set of nerve fibers comprises a diameter(s) that is from about 0.2 μm to about 17.5 μm. In some embodiments, the target nerve fiber or set of nerve fibers comprises a diameter(s) that is from about 0.2 μm to about 17.0 μm. In some embodiments, the target nerve fiber or set of nerve fibers comprises a diameter(s) that is from about 0.2 μm to about 16.5 μm. In some embodiments, the target nerve fiber or set of nerve fibers comprises a diameter(s) that is from about 0.2 μm to about 16.0 μm. In some embodiments, the target nerve fiber or set of nerve fibers comprises a diameter(s) that is from about 0.2 μm to about 15.5 μm. In some embodiments, the target nerve fiber or set of nerve fibers comprises a diameter(s) that is from about 0.2 μm to about 15.0 μm. In some embodiments, the target nerve fiber or set of nerve fibers comprises a diameter(s) that is from about 0.2 μm to about 14.5 μm. In some embodiments, the target nerve fiber or set of nerve fibers comprises a diameter(s) that is from about 0.2 μm to about 14.0 μm. In some embodiments, the target nerve fiber or set of nerve fibers comprises a diameter(s) that is from about 0.2 μm to about 13.5 μm. In some embodiments, the target nerve fiber or set of nerve fibers comprises a diameter(s) that is from about 0.2 μm to about 13.0 μm. In some embodiments, the target nerve fiber or set of nerve fibers comprises a diameter(s) that is from about 0.2 μm to about 12.5 μm. In some embodiments, the target nerve fiber or set of nerve fibers comprises a diameter(s) that is from about 0.2 μm to about 12.0 μm. In some embodiments, the target nerve fiber or set of nerve fibers comprises a diameter(s) that is from about 0.2 μm to about 11.5 μm. In some embodiments, the target nerve fiber or set of nerve fibers comprises a diameter(s) that is from about 0.2 μm to about 11.0 μm. In some embodiments, the target nerve fiber or set of nerve fibers comprises a diameter(s) that is from about 0.2 μm to about 10.5 μm. In some embodiments, the target nerve fiber or set of nerve fibers comprises a diameter(s) that is from about 0.2 μm to about 10.0 μm. In some embodiments, the target nerve fiber or set of nerve fibers comprises a diameter(s) that is from about 0.2 μm to about 9.5 μm. In some embodiments, the target nerve fiber or set of nerve fibers comprises a diameter(s) that is from about 0.2 μm to about 9.0 μm. In some embodiments, the target nerve fiber or set of nerve fibers comprises a diameter(s) that is from about 0.2 μm to about 8.5 μm. In some embodiments, the target nerve fiber or set of nerve fibers comprises a diameter(s) that is from about 0.2 μm to about 8.0 μm. In some embodiments, the target nerve fiber or set of nerve fibers comprises a diameter(s) that is from about 0.2 μm to about 7.5 μm. In some embodiments, the target nerve fiber or set of nerve fibers comprises a diameter(s) that is from about 0.2 μm to about 7.0 μm. In some embodiments, the target nerve fiber or set of nerve fibers comprises a diameter(s) that is from about 0.2 μm to about 6.5 μm. In some embodiments, the target nerve fiber or set of nerve fibers comprises a diameter(s) that is from about 0.2 μm to about 6.0 μm. In some embodiments, the target nerve fiber or set of nerve fibers comprises a diameter(s) that is from about 0.2 μm to about 5.5 μm. In some embodiments, the target nerve fiber or set of nerve fibers comprises a diameter(s) that is from about 0.2 μm to about 5.0 μm. In some embodiments, the target nerve fiber or set of nerve fibers comprises a diameter(s) that is from about 0.2 μm to about 4.5 μm. In some embodiments, the target nerve fiber or set of nerve fibers comprises a diameter(s) that is from about 0.2 μm to about 4.0 μm. In some embodiments, the target nerve fiber or set of nerve fibers comprises a diameter(s) that is from about 0.2 μm to about 3.5 μm. In some embodiments, the target nerve fiber or set of nerve fibers comprises a diameter(s) that is from about 0.2 μm to about 3.0 μm. In some embodiments, the target nerve fiber or set of nerve fibers comprises a diameter(s) that is from about 0.2 μm to about 2.5 μm. In some embodiments, the target nerve fiber or set of nerve fibers comprises a diameter(s) that is from about 0.2 μm to about 2.0 μm. In some embodiments, the target nerve fiber or set of nerve fibers comprises a diameter(s) that is from about 0.2 μm to about 1.5 μm. In some embodiments, the target nerve fiber or set of nerve fibers comprises a diameter(s) that is from about 0.2 μm to about 1.0 μm. In some embodiments, the target nerve fiber or set of nerve fibers comprises a diameter(s) that is from about 0.2 μm to about 0.5 μm. In some embodiments, the target nerve fiber or set of nerve fibers comprises a diameter(s) that is about 0.2 μm.

As would be recognized by one of ordinary skill in the art based on the present disclosure, the diameter of a nerve fiber, including a reference nerve fiber or a target nerve fiber or set of target nerve fibers, can depend on whether the nerve fiber is myelinated or unmyelinated. In some embodiments, the reference nerve fiber is myelinated, and in other embodiments the reference nerve fiber is unmyelinated. In some embodiments, the target nerve fiber or set of nerve fibers is/are myelinated, and in other embodiments the target nerve fiber or set of nerve fibers is/are unmyelinated. In some embodiments, the reference nerve fiber is myelinated, and the target nerve fiber or set of nerve fibers is unmyelinated. In some embodiments, the reference nerve fiber is unmyelinated, and the target nerve fiber or set of nerve fibers is myelinated. In some embodiments, both the reference nerve fiber and the target nerve fiber or set of nerve fibers are myelinated. In some embodiments, both the reference nerve fiber and the target nerve fiber or set of nerve fibers are unmyelinated.

In some embodiments, the hybrid waveform used to selectively block conduction in a target nerve fiber or set of nerve fibers having a diameter(s) that is smaller than a reference nerve fiber comprises a repetition frequency of about 1 kHz to about 200 kHz (as described above). In some embodiments, the hybrid waveform used to selectively block conduction in a target nerve fiber or set of nerve fibers having a diameter(s) that is smaller than a reference nerve fiber comprises a charge imbalance obtained by any of the following, or any combination of the following: (a) adjusting unequally the amplitudes of the phases of the KHF component; (b) adjusting the magnitude of the difference in the phase duration of the KHF component; (c) adjusting the amplitude of the DC offset superimposed on the KHF component; and/or (d) adjusting the shapes of the phases of the KHF components.

In some embodiments, the DC component of the hybrid waveform used to selectively block conduction in a target nerve fiber or set of nerve fibers having a diameter(s) that is smaller than a reference nerve fiber comprises an anodal charge imbalance. In some embodiments, the DC component of the hybrid waveform used to selectively block conduction in a target nerve fiber or set of nerve fibers having a diameter(s) that is smaller than a reference nerve fiber comprises a cathodal charge imbalance. In some embodiments, the DC component comprises an amplitude of greater than or equal to about 1 μA per milliamp of the KHF component per kilohertz of the KHF component. In some embodiments, the DC component comprises an amplitude of greater than or equal to about 1.5 μA per milliamp of the KHF component per kilohertz of the KHF component. In some embodiments, the DC component comprises an amplitude of greater than or equal to about 2.0 μA per milliamp of the KHF component per kilohertz of the KHF component. In some embodiments, the DC component comprises an amplitude of greater than or equal to about 2.5 μA per milliamp of the KHF component per kilohertz of the KHF component. In some embodiments, the DC component comprises an amplitude of greater than or equal to about 3.0 μA per milliamp of the KHF component per kilohertz of the KHF component. In some embodiments, the DC component comprises an amplitude of greater than or equal to about 3.5 μA per milliamp of the KHF component per kilohertz of the KHF component. In some embodiments, the DC component comprises an amplitude of greater than or equal to about 4.0 μA per milliamp of the KHF component per kilohertz of the KHF component. In some embodiments, the DC component comprises an amplitude of greater than or equal to about 4.5 μA per milliamp of the KHF component per kilohertz of the KHF component. In some embodiments, the DC component comprises an amplitude of greater than or equal to about 5.0 μA per milliamp of the KHF component per kilohertz of the KHF component.

In some embodiments, the DC component of the hybrid waveform used to selectively block conduction in a target nerve fiber or set of nerve fibers having a diameter(s) that is smaller than a reference nerve fiber comprises an anodal charge imbalance. In some embodiments, the DC component of the hybrid waveform used to selectively block conduction in a target nerve fiber or set of nerve fibers having a diameter(s) that is smaller than a reference nerve fiber comprises a cathodal charge imbalance. In some embodiments, the DC component comprises an amplitude from about 1 μA to about 100 μA per milliamp of the KHF component per kilohertz of the KHF component. In some embodiments, the DC component comprises an amplitude from about 1 μA to about 90 μA per milliamp of the KHF component per kilohertz of the KHF component. In some embodiments, the DC component comprises an amplitude from about 1 μA to about 80 μA per milliamp of the KHF component per kilohertz of the KHF component. In some embodiments, the DC component comprises an amplitude from about 1 μA to about 70 μA per milliamp of the KHF component per kilohertz of the KHF component. In some embodiments, the DC component comprises an amplitude from about 1 μA to about 60 μA per milliamp of the KHF component per kilohertz of the KHF component. In some embodiments, the DC component comprises an amplitude from about 1 μA to about 50 μA per milliamp of the KHF component per kilohertz of the KHF component. In some embodiments, the DC component comprises an amplitude from about 1 μA to about 40 μA per milliamp of the KHF component per kilohertz of the KHF component. In some embodiments, the DC component comprises an amplitude from about 1 μA to about 30 μA per milliamp of the KHF component per kilohertz of the KHF component. In some embodiments, the DC component comprises an amplitude from about 1 μA to about 20 μA per milliamp of the KHF component per kilohertz of the KHF component. In some embodiments, the DC component comprises an amplitude from about 1 μA to about 10 μA per milliamp of the KHF component per kilohertz of the KHF component. In some embodiments, the DC component comprises an amplitude from about 10 μA to about 100 μA per milliamp of the KHF component per kilohertz of the KHF component. In some embodiments, the DC component comprises an amplitude from about 20 μA to about 100 μA per milliamp of the KHF component per kilohertz of the KHF component. In some embodiments, the DC component comprises an amplitude from about 30 μA to about 100 μA per milliamp of the KHF component per kilohertz of the KHF component. In some embodiments, the DC component comprises an amplitude from about 40 μA to about 100 μA per milliamp of the KHF component per kilohertz of the KHF component. In some embodiments, the DC component comprises an amplitude from about 50 μA to about 100 μA per milliamp of the KHF component per kilohertz of the KHF component. In some embodiments, the DC component comprises an amplitude from about 60 μA to about 100 μA per milliamp of the KHF component per kilohertz of the KHF component. In some embodiments, the DC component comprises an amplitude from about 70 μA to about 100 μA per milliamp of the KHF component per kilohertz of the KHF component. In some embodiments, the DC component comprises an amplitude from about 80 μA to about 100 μA per milliamp of the KHF component per kilohertz of the KHF component. In some embodiments, the DC component comprises an amplitude from about 90 μA to about 100 μA per milliamp of the KHF component per kilohertz of the KHF component.

In some embodiments, the KHF component of the hybrid waveform used to selectively block conduction in a target nerve fiber or set of nerve fibers having a diameter(s) that is smaller than a reference nerve fiber comprises an amplitude between 0.1 mA to 20 mA. In some embodiments, the KHF component comprises an amplitude between 0.5 mA to 20 mA. In some embodiments, the KHF component comprises an amplitude between 1.0 mA to 20 mA. In some embodiments, the KHF component comprises an amplitude between 1.5 mA to 20 mA. In some embodiments, the KHF component comprises an amplitude between 2.0 mA to 20 mA. In some embodiments, the KHF component comprises an amplitude between 2.5 mA to 20 mA. In some embodiments, the KHF component comprises an amplitude between 3.0 mA to 20 mA. In some embodiments, the KHF component comprises an amplitude between 3.5 mA to 20 mA. In some embodiments, the KHF component comprises an amplitude between 4.0 mA to 20 mA. In some embodiments, the KHF component comprises an amplitude between 4.5 mA to 20 mA. In some embodiments, the KHF component comprises an amplitude between 5.0 mA to 20 mA. In some embodiments, the KHF component comprises an amplitude between 5.5 mA to 20 mA. In some embodiments, the KHF component comprises an amplitude between 6.0 mA to 20 mA. In some embodiments, the KHF component comprises an amplitude between 6.5 mA to 20 mA. In some embodiments, the KHF component comprises an amplitude between 7.0 mA to 20 mA. In some embodiments, the KHF component comprises an amplitude between 7.5 mA to 20 mA. In some embodiments, the KHF component comprises an amplitude between 8.0 mA to 20 mA. In some embodiments, the KHF component comprises an amplitude between 8.5 mA to 20 mA. In some embodiments, the KHF component comprises an amplitude between 9.0 mA to 20 mA. In some embodiments, the KHF component comprises an amplitude between 9.5 mA to 20 mA. In some embodiments, the KHF component comprises an amplitude between 10.0 mA to 20 mA. In some embodiments, the KHF component comprises an amplitude between 10.5 mA to 20 mA. In some embodiments, the KHF component comprises an amplitude between 11.0 mA to 20 mA. In some embodiments, the KHF component comprises an amplitude between 11.5 mA to 20 mA. In some embodiments, the KHF component comprises an amplitude between 12.0 mA to 20 mA. In some embodiments, the KHF component comprises an amplitude between 12.5 mA to 20 mA. In some embodiments, the KHF component comprises an amplitude between 13.0 mA to 20 mA. In some embodiments, the KHF component comprises an amplitude between 13.5 mA to 20 mA. In some embodiments, the KHF component comprises an amplitude between 14.0 mA to 20 mA. In some embodiments, the KHF component comprises an amplitude between 14.5 mA to 20 mA. In some embodiments, the KHF component comprises an amplitude between 15.0 mA to 20 mA. In some embodiments, the KHF component comprises an amplitude between 15.5 mA to 20 mA. In some embodiments, the KHF component comprises an amplitude between 16.0 mA to 20 mA. In some embodiments, the KHF component comprises an amplitude between 16.5 mA to 20 mA. In some embodiments, the KHF component comprises an amplitude between 17.0 mA to 20 mA. In some embodiments, the KHF component comprises an amplitude between 17.5 mA to 20 mA. In some embodiments, the KHF component comprises an amplitude between 18.0 mA to 20 mA. In some embodiments, the KHF component comprises an amplitude between 18.5 mA to 20 mA. In some embodiments, the KHF component comprises an amplitude between 19.0 mA to 20 mA. In some embodiments, the KHF component comprises an amplitude between 19.5 mA to 20 mA. In some embodiments, the KHF component comprises an amplitude between 1.0 mA to 15 mA. In some embodiments, the KHF component comprises an amplitude between 5.0 mA to 10 mA. In some embodiments, the KHF component comprises an amplitude between 0.5 mA to 5 mA. In some embodiments, the KHF component comprises an amplitude between 10.0 mA to 20.0 mA.

In accordance with the above, embodiments of the present disclosure also includes a hybrid waveform that blocks conduction in a target nerve fiber or set of nerve fibers comprising a diameter(s) that is larger than a reference nerve fiber, but does not block conduction in the reference nerve. In some embodiments, the reference nerve fiber comprises a diameter that is from about 0.2 μm to about 19.5 μm. In some embodiments, the reference nerve fiber comprises a diameter that is from about 0.5 μm to about 19.5 μm. In some embodiments, the reference nerve fiber comprises a diameter that is from about 1.0 μm to about 19.5 μm. In some embodiments, the reference nerve fiber comprises a diameter that is from about 1.5 μm to about 19.5 μm. In some embodiments, the reference nerve fiber comprises a diameter that is from about 2.0 μm to about 19.5 μm. In some embodiments, the reference nerve fiber comprises a diameter that is from about 2.5 μm to about 19.5 μm. In some embodiments, the reference nerve fiber comprises a diameter that is from about 3.0 μm to about 19.5 μm. In some embodiments, the reference nerve fiber comprises a diameter that is from about 3.5 μm to about 19.5 μm. In some embodiments, the reference nerve fiber comprises a diameter that is from about 4.0 μm to about 19.5 μm. In some embodiments, the reference nerve fiber comprises a diameter that is from about 4.5 μm to about 19.5 μm. In some embodiments, the reference nerve fiber comprises a diameter that is from about 5.0 μm to about 19.5 μm. In some embodiments, the reference nerve fiber comprises a diameter that is from about 5.5 μm to about 19.5 μm. In some embodiments, the reference nerve fiber comprises a diameter that is from about 6.0 μm to about 19.5 μm. In some embodiments, the reference nerve fiber comprises a diameter that is from about 6.5 μm to about 19.5 μm. In some embodiments, the reference nerve fiber comprises a diameter that is from about 7.0 μm to about 19.5 μm. In some embodiments, the reference nerve fiber comprises a diameter that is from about 7.5 μm to about 19.5 μm. In some embodiments, the reference nerve fiber comprises a diameter that is from about 8.0 μm to about 19.5 μm. In some embodiments, the reference nerve fiber comprises a diameter that is from about 8.5 μm to about 19.5 μm. In some embodiments, the reference nerve fiber comprises a diameter that is from about 9.0 μm to about 19.5 μm. In some embodiments, the reference nerve fiber comprises a diameter that is from about 9.5 μm to about 19.5 μm. In some embodiments, the reference nerve fiber comprises a diameter that is from about 10.0 μm to about 19.5 μm. In some embodiments, the reference nerve fiber comprises a diameter that is from about 10.5 μm to about 19.5 μm. In some embodiments, the reference nerve fiber comprises a diameter that is from about 11.0 μm to about 19.5 μm. In some embodiments, the reference nerve fiber comprises a diameter that is from about 11.5 μm to about 19.5 μm. In some embodiments, the reference nerve fiber comprises a diameter that is from about 12.0 μm to about 19.5 μm. In some embodiments, the reference nerve fiber comprises a diameter that is from about 12.5 μm to about 19.5 μm. In some embodiments, the reference nerve fiber comprises a diameter that is from about 13.0 μm to about 19.5 μm. In some embodiments, the reference nerve fiber comprises a diameter that is from about 13.5 μm to about 19.5 μm. In some embodiments, the reference nerve fiber comprises a diameter that is from about 14.0 μm to about 19.5 μm. In some embodiments, the reference nerve fiber comprises a diameter that is from about 14.5 μm to about 19.5 μm. In some embodiments, the reference nerve fiber comprises a diameter that is from about 15.0 μm to about 19.5 μm. In some embodiments, the reference nerve fiber comprises a diameter that is from about 15.5 μm to about 19.5 μm. In some embodiments, the reference nerve fiber comprises a diameter that is from about 16.0 μm to about 19.5 μm. In some embodiments, the reference nerve fiber comprises a diameter that is from about 16.5 μm to about 19.5 μm. In some embodiments, the reference nerve fiber comprises a diameter that is from about 17.0 μm to about 19.5 μm. In some embodiments, the reference nerve fiber comprises a diameter that is from about 17.5 μm to about 19.5 μm. In some embodiments, the reference nerve fiber comprises a diameter that is from about 18.0 μm to about 19.5 μm. In some embodiments, the reference nerve fiber comprises a diameter that is from about 18.5 μm to about 19.5 μm. In some embodiments, the reference nerve fiber comprises a diameter that is from about 19.0 μm to about 19.5 μm.

In accordance with the above embodiments, the target nerve fiber or set of nerve fibers is larger than the reference nerve fiber and comprises a diameter(s) from about 0.5 μm to about 20.0 μm. In some embodiments, the target nerve fiber or set of nerve fibers comprises a diameter(s) that is from about 0.5 μm to about 19.0 μm. In some embodiments, the target nerve fiber or set of nerve fibers comprises a diameter(s) that is from about 0.5 μm to about 18.5 μm. In some embodiments, the target nerve fiber or set of nerve fibers comprises a diameter(s) that is from about 0.5 μm to about 18.0 μm. In some embodiments, the target nerve fiber or set of nerve fibers comprises a diameter(s) that is from about 0.5 μm to about 17.5 μm. In some embodiments, the target nerve fiber or set of nerve fibers comprises a diameter(s) that is from about 0.5 μm to about 17.0 μm. In some embodiments, the target nerve fiber or set of nerve fibers comprises a diameter(s) that is from about 0.5 μm to about 16.5 μm. In some embodiments, the target nerve fiber or set of nerve fibers comprises a diameter(s) that is from about 0.5 μm to about 16.0 μm. In some embodiments, the target nerve fiber or set of nerve fibers comprises a diameter(s) that is from about 0.5 μm to about 15.5 μm. In some embodiments, the target nerve fiber or set of nerve fibers comprises a diameter(s) that is from about 0.5 μm to about 15.0 μm. In some embodiments, the target nerve fiber or set of nerve fibers comprises a diameter(s) that is from about 0.5 μm to about 14.5 μm. In some embodiments, the target nerve fiber or set of nerve fibers comprises a diameter(s) that is from about 0.5 μm to about 14.0 μm. In some embodiments, the target nerve fiber or set of nerve fibers comprises a diameter(s) that is from about 0.5 μm to about 13.5 μm. In some embodiments, the target nerve fiber or set of nerve fibers comprises a diameter(s) that is from about 0.5 μm to about 13.0 μm. In some embodiments, the target nerve fiber or set of nerve fibers comprises a diameter(s) that is from about 0.5 μm to about 12.5 μm. In some embodiments, the target nerve fiber or set of nerve fibers comprises a diameter(s) that is from about 0.5 μm to about 12.0 μm. In some embodiments, the target nerve fiber or set of nerve fibers comprises a diameter(s) that is from about 0.5 μm to about 11.5 μm. In some embodiments, the target nerve fiber or set of nerve fibers comprises a diameter(s) that is from about 0.5 μm to about 11.0 μm. In some embodiments, the target nerve fiber or set of nerve fibers comprises a diameter(s) that is from about 0.5 μm to about 10.5 μm. In some embodiments, the target nerve fiber or set of nerve fibers comprises a diameter(s) that is from about 0.5 μm to about 10.0 μm. In some embodiments, the target nerve fiber or set of nerve fibers comprises a diameter(s) that is from about 0.5 μm to about 9.5 μm. In some embodiments, the target nerve fiber or set of nerve fibers comprises a diameter(s) that is from about 0.5 μm to about 9.0 μm. In some embodiments, the target nerve fiber or set of nerve fibers comprises a diameter(s) that is from about 0.5 μm to about 8.5 μm. In some embodiments, the target nerve fiber or set of nerve fibers comprises a diameter(s) that is from about 0.5 μm to about 8.0 μm. In some embodiments, the target nerve fiber or set of nerve fibers comprises a diameter(s) that is from about 0.5 μm to about 7.5 μm. In some embodiments, the target nerve fiber or set of nerve fibers comprises a diameter(s) that is from about 0.5 μm to about 7.0 μm. In some embodiments, the target nerve fiber or set of nerve fibers comprises a diameter(s) that is from about 0.5 μm to about 6.5 μm. In some embodiments, the target nerve fiber or set of nerve fibers comprises a diameter(s) that is from about 0.5 μm to about 6.0 μm. In some embodiments, the target nerve fiber or set of nerve fibers comprises a diameter(s) that is from about 0.5 μm to about 5.5 μm. In some embodiments, the target nerve fiber or set of nerve fibers comprises a diameter(s) that is from about 0.5 μm to about 5.0 μm. In some embodiments, the target nerve fiber or set of nerve fibers comprises a diameter(s) that is from about 0.5 μm to about 4.5 μm. In some embodiments, the target nerve fiber or set of nerve fibers comprises a diameter(s) that is from about 0.5 μm to about 4.0 μm. In some embodiments, the target nerve fiber or set of nerve fibers comprises a diameter(s) that is from about 0.5 μm to about 3.5 μm. In some embodiments, the target nerve fiber or set of nerve fibers comprises a diameter(s) that is from about 0.5 μm to about 3.0 μm. In some embodiments, the target nerve fiber or set of nerve fibers comprises a diameter(s) that is from about 0.5 μm to about 2.5 μm. In some embodiments, the target nerve fiber or set of nerve fibers comprises a diameter(s) that is from about 0.5 μm to about 2.0 μm. In some embodiments, the target nerve fiber or set of nerve fibers comprises a diameter(s) that is from about 0.5 μm to about 1.5 μm. In some embodiments, the target nerve fiber or set of nerve fibers comprises a diameter(s) that is from about 0.5 μm to about 1.0 μm.

In some embodiments, the hybrid waveform used to selectively block conduction in a target nerve fiber or set of nerve fibers having a diameter(s) that is larger than a reference nerve fiber comprises a repetition frequency of about 1 kHz to about 200 kHz. In some embodiments, the hybrid waveform used to selectively block conduction in a target nerve fiber or set of nerve fibers having a diameter(s) that is larger than a reference nerve fiber comprises a charge imbalance obtained by any of the following, or any combination of the following: (a) adjusting unequally the amplitudes of the phases of the KHF component; (b) adjusting the magnitude of the difference in the phase duration of the KHF component; (c) adjusting the amplitude of the DC offset superimposed on the KHF component; and/or (d) adjusting the shapes of the phases of the KHF components.

In some embodiments, the DC component of the hybrid waveform used to selectively block conduction in a target nerve fiber or set of nerve fibers having a diameter(s) that is larger than a reference nerve fiber comprises an anodal charge imbalance. In some embodiments, the DC component of the hybrid waveform used to selectively block conduction in a target nerve fiber or set of nerve fibers having a diameter(s) that is larger than a reference nerve fiber comprises a cathodal charge imbalance. In some embodiments, the DC component comprises an amplitude of 0 μA to 100 μA per milliamp of KHF component per kilohertz of the KHF component. In some embodiments, the KHF component comprises an amplitude of 0.1 mA to 20 mA.

Regardless of whether the target nerve fiber or set of nerve fibers is smaller or larger than a reference nerve fiber, embodiments of the present disclosure include methods for blocking nerve fiber conduction in a unidirectional manner. In some embodiments, the method for selective nerve fiber conduction includes adjusting polarity of the DC component. In some embodiments, adjusting the polarity of the DC component includes reversing the polarity of the DC component such that conduction can be blocked in a unidirectional manner. In some embodiments, adjusting the polarity of the DC component includes using one or more electrical contacts (e.g., electrodes) with respect to the target nerve fiber or set of nerve fibers. In some embodiments, adjusting the polarity of the DC component includes using two or more electrical contacts (e.g., electrodes) with respect to the target nerve fiber or set of nerve fibers.

In accordance with these embodiments, the hybrid waveform used to obtain unidirectional conduction block can comprise a repetition frequency of about 1 kHz to about 200 kHz. In some embodiments, the hybrid waveform can comprise a charge imbalance obtained by of the following or any combination of the following: (a) adjusting unequally the amplitudes of the phases of the KHF component; (b) adjusting the magnitude of the difference in the phase duration of the KHF component; (c) adjusting the amplitude of the DC offset superimposed on the KHF component; and/or (d) adjusting the shapes of the phases of the KHF components. In some embodiments, the DC component of the hybrid waveform capable of achieving unidirectional conduction block comprises an anodal charge imbalance. In some embodiments, the DC component of the hybrid waveform capable of achieving unidirectional conduction block comprises a cathodal charge imbalance. In some embodiments, the DC component of the hybrid waveform capable of achieving unidirectional conduction block comprises an amplitude of greater than or equal to about 1 μA per milliamp of the KHF component per kilohertz of the KHF component. In some embodiments, the KHF component of the hybrid waveform capable of achieving unidirectional conduction block comprises an amplitude between 0.1 mA to 20 mA.

### 3. METHODS AND SYSTEMS

Embodiments of the present disclosure also include a system for selective nerve fiber conduction block. In accordance with these embodiments, the system includes an electrode with one or more metal contacts sized and configured for implantation in proximity to neural tissue, and a pulse generator coupled to the electrode, the pulse generator including a power source comprising a battery and a microprocessor coupled to the battery. In some embodiments, the pulse generator is capable of applying to the electrode a hybrid waveform capable of achieving selective conduction block in a target nerve fiber or set of nerve fibers.

As described further herein, the hybrid waveform applied to a subject using a neuromodulation system comprises a KHF component comprising a biphasic alternating current waveform, and a DC component obtained by: (a) adjusting unequally the amplitudes of the phases of the KHF component; (b) adjusting the magnitude of the difference in the phase duration of the KHF component; (c) adjusting the amplitude of the DC offset superimposed on the KHF component; and/or (d) adjusting the shapes of the phases of the KHF components; and any combinations of (a)-(d). In some embodiments, the hybrid waveform comprises a net charge imbalance per unit time. In some embodiments, the hybrid waveform blocks conduction in the target nerve fiber or set of nerve fibers but does not block conduction in a reference nerve fiber or set of nerve fibers.

Embodiments of the present disclosure also include a method for obtaining selective nerve fiber conduction block in a subject using any of the systems described herein. In some embodiments, the method includes programming the pulse generator to output the hybrid waveform such that the hybrid waveform blocks neural conduction when delivered by the pulse generator. In some embodiments, the KHF component comprises a biphasic alternating current waveform. In some embodiments, the KHF component comprises a waveform with more than two phases. In some embodiments, the DC component comprises a DC offset superimposed on the KHF component. In some embodiments, the DC component comprises unequal phase durations or unequal amplitudes of phases in the KHF component. In some embodiments, the hybrid waveform is repeated at a frequency of about 1 kHz to about 200 kHz.

In some embodiments, the hybrid waveform applied to a subject using a neuromodulation system comprises a charge imbalance obtained by: (a) adjusting unequally the amplitudes of the phases of the KHF component; (b) adjusting the magnitude of the difference in the phase duration of the KHF component; (c) adjusting the amplitude of the DC offset superimposed on the KHF component; and/or (d) adjusting the shapes of the phases of the KHF components; and any combinations of (a)-(d). In some embodiments, the DC component comprises an anodal charge imbalance or a cathodal charge imbalance. In some embodiments, the DC component comprises an amplitude of greater than or equal to about 1 μA per milliamp of the KHF component per kilohertz of the KHF component. In some embodiments, the KHF component comprises an amplitude between 0.1 mA to 20 mA. In some embodiments, the hybrid waveform blocks conduction in the target nerve fiber or set of nerve fibers but does not block conduction in a reference nerve fiber or set of nerve fibers.

Embodiments of the present disclosure also include a method for obtaining unidirectional nerve fiber conduction block in a subject using a neuromodulation device. In accordance with these embodiments, the method includes applying a hybrid waveform comprising a kilohertz frequency (KHF) component and a direct current (DC) component to a target nerve fiber or set of nerve fibers, such that the hybrid waveform achieves a conduction block in the target nerve fiber or set of nerve fibers in a unidirectional manner.

Embodiments of the present disclosure also include a system for obtaining unidirectional nerve fiber conduction block in a subject. In accordance with these embodiments, the system includes an electrode with one or more metal contacts sized and configured for implantation in proximity to neural tissue, and a pulse generator coupled to the electrode, the pulse generator including a power source comprising a battery and a microprocessor coupled to the battery, such that the pulse generator is capable of applying to the electrode a hybrid waveform capable of achieving unidirectional conduction block in a target nerve fiber or set of nerve fibers.

In some embodiments, the hybrid waveform applied to a subject using a neuromodulation system comprises a KHF component comprising a biphasic alternating current waveform, and a DC component obtained by: (a) adjusting unequally the amplitudes of the phases of the KHF component; (b) adjusting the magnitude of the difference in the phase duration of the KHF component; (c) adjusting the amplitude of the DC offset superimposed on the KHF component; and/or (d) adjusting the shapes of the phases of the KHF components; and any combinations of (a)-(d). In some embodiments, the hybrid waveform comprises a net charge imbalance per unit time. In some embodiments, the hybrid waveform blocks conduction in the target nerve fiber or set of nerve fibers but does not block conduction in a reference nerve fiber or set of nerve fibers.

Embodiments of the present disclosure also include a method for obtaining unidirectional nerve fiber conduction block in a subject using any of the systems described herein. In some embodiments, the method includes programming the pulse generator to output the hybrid waveform such that the hybrid waveform blocks neural conduction in a unidirectional manner when delivered by the pulse generator.

In accordance with the systems and methods described above, embodiments of the present disclosure include programming a pulse generator to output the hybrid waveform (e.g., on a graphical user interface (GUI)), the hybrid waveform capable of selectively blocking neural conduction, and setting the amplitude of the waveform such that the waveform blocks neural conduction when delivered by the pulse generator.

In some embodiments, the systems/methods for selectively blocking neural conduction as described herein include placing one or more electrodes or leads in a desired position in contact with nervous system tissue of a subject receiving neural block conduction treatment. In some embodiments, the electrode(s) can be implanted in a region of the brain. In other embodiments, the electrode(s) can be implanted in, on, or near the spinal cord; or in, on, or near a peripheral nerve (sensory or motor or mixed; somatic or autonomic); or in, or, or near a neural plexus; or in, on, or near any subcutaneous tissue such as muscle tissue (including cardiac tissue) or adipose tissue or other organ tissue to achieve a particular therapeutic purpose.

The electrode can be one or more electrodes configured as part of the distal end of a lead or be one or more electrodes configured as part of a leadless system to apply electrical pulses to the targeted tissue region. Electrical pulses can be supplied by a pulse generator coupled to the electrode/lead. In one embodiment, the pulse generator can be implanted in a suitable location remote from the electrode/lead (e.g., in the shoulder region); however, that the pulse generator could be placed in other regions of the body or externally to the body.

When implanted, at least a portion of the case or housing of the pulse generator can serve as a reference or return electrode. Alternatively, the lead can include a reference or return electrode (comprising a multipolar (such as bipolar) arrangement), or a separate reference or return electrode can be implanted or attached elsewhere on the body (comprising a monopolar arrangement).

The pulse generator can include stimulation generation circuitry, which can include an on-board, programmable microprocessor, which has access to and/or carries embedded code. The code expresses pre-programmed rules or algorithms under which desired electrical stimulation is generated, having desirable electrical stimulation parameters that may also be calculated by the microprocessor, and distributed to the electrode(s) on the lead. According to these programmed rules, the pulse generator directs the stimulation through the lead to the electrode(s), which serve to selectively stimulate the targeted tissue region. The code may be programmed, altered or selected by a clinician to achieve the particular physiologic response desired. Additionally or alternatively to the microprocessor, stimulation generation circuitry may include discrete electrical components operative to generate electrical stimulation having desirable parameters for blocking neural conduction. As described herein, the parameters can be input to generate any of the hybrid waveforms of the present disclosure. One or more of the parameters may be prescribed or predetermined as associated with a particular treatment regime or indication (e.g., to reduce pain). In some embodiments, the pulse generator can be programmed to output a hybrid waveform (e.g., on a graphical user interface (GUI)), and the waveform can be capable of blocking neural conduction, as described further herein.

### 4. EXAMPLES

It will be readily apparent to those skilled in the art that other suitable modifications and adaptations of the methods of the present disclosure described herein are readily applicable and appreciable, and may be made using suitable equivalents without departing from the scope of the present disclosure or the aspects and embodiments disclosed herein. Having now described the present disclosure in detail, the same will be more clearly understood by reference to the following examples, which are merely intended only to illustrate some aspects and embodiments of the disclosure, and should not be viewed as limiting to the scope of the disclosure. The disclosures of all journal references, U.S. patents, and publications referred to herein are hereby incorporated by reference in their entireties.

The present disclosure has multiple aspects, illustrated by the following non-limiting examples.

### Example 1

Using a computational model of the rat tibial nerve and in vivo recordings of rat gastrocnemius muscle force, the effects of charge imbalance, frequency, and asymmetry of KHF signals on block thresholds were quantified across a suite of biphasic rectangular KHF waveforms mixed with different levels of DC. All data analyses and statistics were conducted in MATLAB R2018a (Mathworks; Natick, Mass.).

The effects of DC offset on block thresholds measured in vivo using the following mathematical model:

$$T = {T}_{0} {e}^{- m \vert L \vert f / ( {L}_{ma x} * {f}_{m ax} )} \qquad ( Equation 1 )$$

where T is the block threshold of a waveform with a DC offset, To is the block threshold of the same waveform without a DC offset, f is the frequency in kilohertz, L is the level of amplitude- and frequency-dependent DC offset in μA DC per mA KHF per 1 kHz, m is a coefficient to be fit, Lₘₐₓ is the maximum DC offset level evaluated in μA DC per mA KHF per 1 kHz, and fₘₐₓ is the maximum frequency evaluated in kilohertz. In the presence of a non-zero DC offset, for the KHF signals with amplitude- and frequency-dependent DC offsets that were evaluated in vivo, Equation 1 specifies that block threshold decays toward zero as DC offset or frequency increase. The mathematical model was further extended with three additional variables to account for the presence of two distinct DC offset polarities and for the fact that repeated measures were obtained of each nerve and each frequency:

$$T = {p}_{i} {a}_{j} {c}_{k} {T}_{0} {e}^{- m \vert L \vert f / ( {L}_{m ax} * {f}_{m ax} )} \qquad ( Equation 2 )$$

Parameters pᵢ, aⱼ, and cₖ were adjustment factors for a specific polarity i, a specific nerve j, and a specific frequency k, respectively. Lₘₐₓ was set to 4 μA DC per mA KHF per 1 kHz, set fₘₐₓ to 80 kHz, and took the natural log of both sides of the Equation 2 to produce the following linear equation:

$$ln T = ln {p}_{i} + ln {a}_{j} + ln {c}_{k} + ln {T}_{0} - m * \frac{\vert L \vert f}{4 * 80} \qquad ( Equation 3 )$$

Equation 3 was fit to in vivo data quantifying block for symmetric waveforms with DC offsets using a three-way ANCOVA with one covariate (anovan function in MATLAB R2018a, setting polarity, nerve index, and frequency as categorical grouping variables, and DC offset as a continuous variable). Equation 3 was also separately fit to measurements of charge imbalance effects due to asymmetric waveforms. Approximate normality of residuals was verified using Q-Q plots and residual histograms, and results of Anderson-Darling tests were reported for normality.

### Example 2

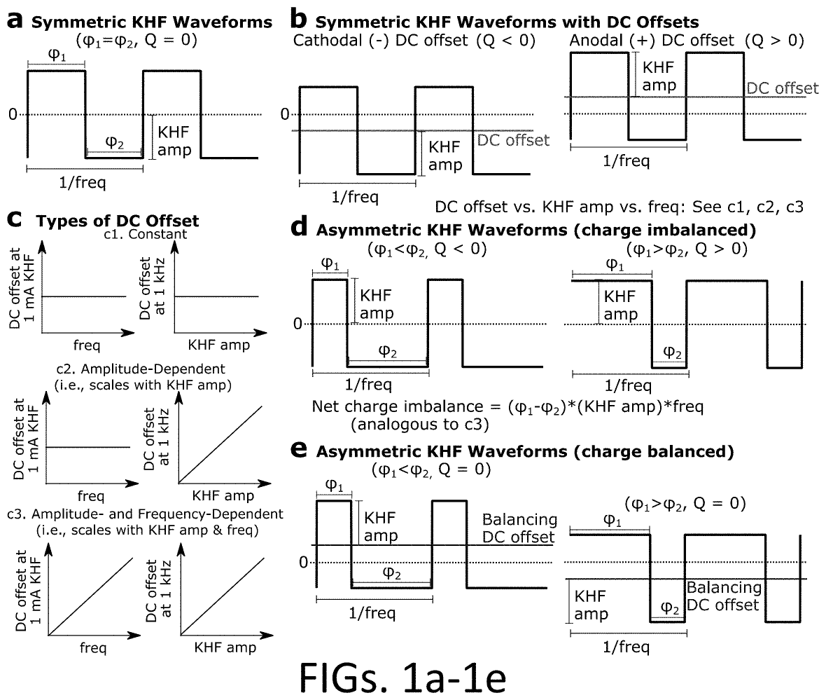

Nerve Block Waveforms. A suite of rectangular waveforms were evaluated in computational models and in vivo (1) to identify the properties of nerve block instrumentation that could lead to non-monotonic block thresholds, and (2) to probe the mechanisms of non-monotonic block thresholds by disentangling the individual contributions of waveform components to block thresholds across frequencies. In computational models, the type of DC offset important for non-monotonic block thresholds was probed by comparing symmetric rectangular waveforms with zero net charge (FIG. 1a) against symmetric rectangular waveforms with added or subtracted DC offsets (FIG. 1b), where “symmetry” refers to equal duration phases. Three different types of DC offset were evaluated, corresponding to hypothetical nerve block instruments with distinct dependencies between a KHF signal and unintended DC offsets (FIG. 1c, subpanels c1, c2, c3): (1) “constant DC offset” that was independent of any KHF parameter; (2) “amplitude-dependent DC offset” that scaled linearly with KHF amplitude; (3) “amplitude- and frequency-dependent DC offset” that scaled linearly with both KHF amplitude and frequency. KHF amplitude was defined as half of the peak-to-peak amplitude in all cases (FIG. 1b). Constant DC offset values were ±15, ±26, ±46, ±80, ±106, ±141, ±186, ±246, ±326, ±431, ±754, and ±1,320 μA. Amplitude-dependent DC offset values were ±10, ±20, ±40, ±59, ±77, ±100, ±125, ±143, ±167, ±200, and ±400 μA per mA of KHF. Amplitude- and frequency-dependent DC offset values were ±0.5, ±1, ±1.5, ±2, ±2.5, ±3, ±3.5, and ±4 μA per mA of KHF per 1 kHz. The choice of DC offsets was based on preliminary simulations, and spanned the relevant range of values such that the smallest offsets had little or no effect while the largest offsets had a saturated or nearly saturated effect. All waveforms were evaluated at 10, 20, 29.4, 38.5, 50, 62.5, 71.4, 83.3, and 100 kHz. These frequencies had periods that were integer multiples of 1 μs to ensure that waveform discretization in computational models resulted only in the intended amounts of charge imbalance.

Previous computational modeling studies evaluated block thresholds of asymmetric rectangular waveforms, corresponding to hypothetical nerve block instruments that generate waveforms with unintended asymmetry. While such waveforms produced non-monotonic block thresholds, the individual contributions of asymmetry and charge imbalance were unclear. Therefore, two types of asymmetric waveforms were evaluated that—along with tests of symmetric waveforms with DC offsets—enabled analysis of individual contributions of asymmetry and charge imbalance to non-monotonic block thresholds. The first type of asymmetric waveform replicated the asymmetry from the previous study (FIG. 1d), such that the differences in duration between the first and second phases (in μs) were constant across all frequencies and thus produced net charge per unit time (Q), i.e., DC, that scaled with KHF amplitude and frequency, similar to that illustrated in FIG. 1c, subpanel c3. A phase difference of 1 μs produced equivalent net charge per unit time (Q) as that produced by an amplitude- and frequency-dependent DC offset of 1 μA DC per mA KHF per 1 kHz. The second type of asymmetric waveform was constructed from the first type with a compensatory DC offset that resulted in zero net charge per unit time (FIG. 1e). Computational models were simulated at the same frequencies as the symmetric waveforms described above to evaluate block thresholds for both types of asymmetric waveforms with phase differences of ±2, ±3, and ±4 μs.

In vivo experiments were conducted to validate the predictions from computational models of symmetric waveforms without DC offsets (FIG. 1a), symmetric waveforms with DC offsets (FIG. 1b) that were amplitude- and frequency-dependent (FIG. 1c, subpanel c3), and asymmetric waveforms that were charge-imbalanced (FIG. 1d) and charge-balanced (FIG. 1e). The symmetric waveforms with DC offsets were offset by ±2, ±3, ±4 μA DC per mA KHF per 1 kHz. Phase differences for asymmetric waveforms were ±2, ±3, and ±4 μs, enabling direct comparison between DC offset symmetric waveforms and charge-imbalanced asymmetric waveforms. The phase differences, in turn, were in a range similar to previous modeling work on asymmetric charge-imbalanced waveforms, facilitating comparisons of the present symmetric and asymmetric work to previous studies. All waveforms were evaluated in vivo at 20, 40, 60, and 80 kHz.

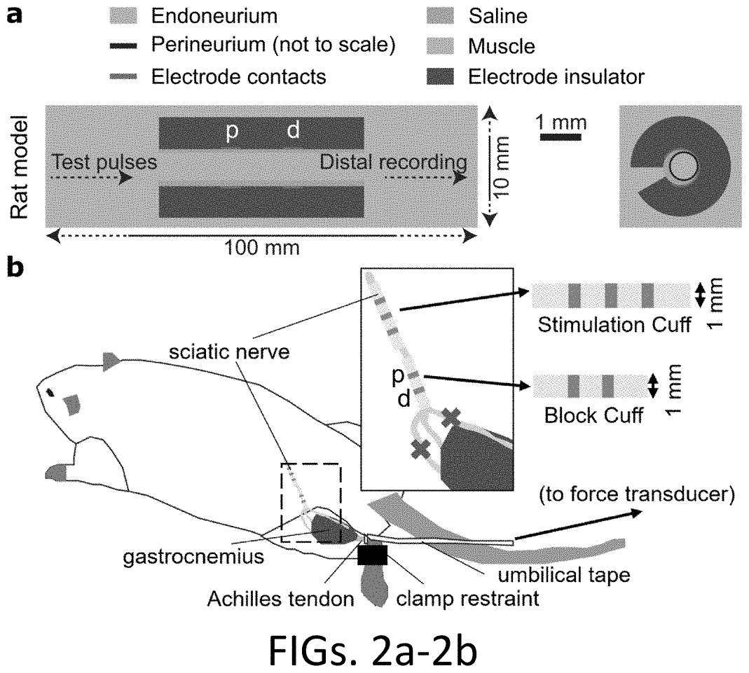

All waveforms were evaluated at positive and negative polarities, corresponding to positive or negative DC offsets or phase differences (FIGS. 1b, 1d, 1e). Unless otherwise specified, polarity was referred to in terms of the proximal contact of the bipolar blocking electrode, such that negative (or cathodal) DC and positive (or anodal) DC correspond to current sinks and current sources at the proximal electrode contact, respectively (see FIG. 2 and corresponding Methods text for electrode orientation details).

### Example 3

Computational Model—Finite Element Models of Rat Tibial Nerve. A finite element model (FEM) of a rat tibial nerve and cuff electrode was implemented using COMSOL Multiphysics v5.3a (Burlington, Mass.) (FIG. 2a). The monofascicular rat tibial nerve was modeled as a 0.75 mm diameter cylinder surrounded by a bipolar cuff electrode (contacts 0.5 mm in length spaced 1 mm edge-to-edge; 1.5 mm between each edge of the cuff and the nearest contact edge; 5 mm total cuff length; 0.875 mm insulator thickness; 1 mm inner diameter); the insulator surrounded 330° of the nerve circumferentially and the contacts spanned 270°. The nerve was positioned 10 μm away from the inner wall of the cuff that was opposite the cuff opening, and the cuff was centered along the length of the 100 mm-long nerve. A point current source was placed within each of the platinum ribbon electrode domains (+1 mA in the proximal contact and −1 mA in the distal contact), in accordance with a methods study on modeling current sources for neural stimulation in COMSOL. All outermost surfaces of the model were grounded except the ends of the nerve. The insulator of the cuff was modeled as silicone (1e12 Ω-m²⁷) and the contacts were modeled as platinum (1.06e-7 Ω-m). The endoneurium was modeled as an anisotropic medium (1.75 Ω-m longitudinally, 6 Ω-m radially), the perineurium using a thin layer approximation (COMSOL's contact impedance boundary condition; thickness equal to 3% of the fascicle diameter; 1149 Ω-m), the space between the nerve and the cuff as isotropic saline (0.568 Ω-m), and the rest of the tissue outside the nerve and cuff as anisotropic muscle (2.86 Ω-m longitudinally, 11.6 Ω-m radially; 10 mm diameter).

The 100 mm-long FEM was meshed with 1,510,090 tetrahedral elements. Quadratic geometry and solution shape functions, and the conjugate gradients solver were used to solve Laplace's equation for potentials in the volume assuming quasi-static conditions and non-dispersive materials. The mesh density was doubled until the block threshold for a 10 kHz symmetric rectangular wave with zero offset applied to a 100 mm-long, 5.7 μm diameter axon at the center of the nerve changed <3%.

Computational Model—Simulations of Biophysical Axons. The electric potentials were applied from the FEM to 100 mm-long model axons centered in the nerve. Mammalian myelinated axons were stimulated using the McIntyre-Richardson-Grill (MRG) model in NEURON v7.5. Approximately 5.7 μm-diameter axons were used for most simulations and 5.7, 7.3, 8.7, 10, and 11.5 μm-diameter axons for the comparisons of effects across fiber diameters. The chosen range of fiber diameters is representative of those reported for rat tibial nerve. Passive end nodes were included to reduce edge effects (gₘ=0.0001 S/cm², cm=2 μF/cm², −70 mV reversal potential). The middle node of Ranvier of each axon was aligned with the middle of the FEM.

Each simulation was initialized with 10 ms time steps from t=−200 ms to t=0 ms to ensure initial steady-state and ran each simulation from t=0 ms to t=250 ms with 0.5 μs time steps (backward Euler integration). Supra-threshold 2 nA intracellular test pulses were delivered every 50 ms starting at t=25 ms at the node of Ranvier closest to 6 mm from the proximal end of the nerve. The KHF waveform was delivered starting at t=1 ms. For each KHF waveform, the potentials obtained from the FEM were scaled to simulate amplitudes from 0.05 to 5 mA in 6% increments. The action potentials were counted at the node of Ranvier closest to 12 mm from the distal end of the nerve starting at t=100 ms, which allowed sufficient time for the onset response to subside. “Transmission”, “block”, and “excitation” were defined in terms of recorded action potentials between 100 and 250 ms. “Transmission” was the presence of exactly three action potentials spaced 50 ms apart (1 ms tolerance) in response to the test pulses at t=125, 175, and 225 ms, with the first action potential occurring within 5 ms of a test pulse (i.e., allowing for conduction delay). “Block” was the total absence of action potentials after t=100 ms. “Excitation” was anything that was neither “transmission” nor “block”. “Block threshold” was the minimum amplitude that produced block. To prevent spurious block threshold measurements in computational models, block was maintained at least 0.1 mA above block threshold, except in two simulations with block windows that were truly smaller than 0.1 mA (i.e., symmetric rectangular waves at 10 kHz with +167 and +200 μA DC offset per mA KHF).

### Example 4

The In Vivo Electrical Block of the Rat Tibial Nerve. Acute experiments were conducted to quantify in vivo responses of the tibial nerve to KHF signals in male Sprague-Dawley rats (n=7; 362 to 678 g, median=440 g; Charles River Laboratories) by recording the force generated by the gastrocnemius (FIG. 2b). All procedures were approved by the Institute for Animal Care and Use Committee of Duke University (Durham, N.C.) and were in accordance with the Guide for Care and Use of Laboratory Animals (8th edition). The study was also carried out in compliance with the ARRIVE guidelines. The animals were housed under USDA- and AAALAC-compliant conditions, with 12 h/12 h light/dark cycle and free access to food, water, and environmental enrichment. Rats were placed in an anesthesia box, briefly anesthetized with 3% isoflurane in air, and then injected subcutaneously with 1.2 g/kg urethane, with supplemental doses administered as required (up to 0.4 g/kg total; SQ, IM, or IP). Heart rate and blood oxygenation were monitored continuously using a pulse oximeter (PalmSAT 2500A; Nonin Medical; Plymouth, Minn., USA), and depth of anesthesia was assessed using the toe pinch reflex and heart rate. Body temperature was monitored using a rectal temperature probe (TH-8 Thermalert; Physitemp Instruments, Inc.; Clifton, N.J.) and maintained between ˜35-38° C. with a heated water blanket.

The surgical methods described in a prior publication were adapted to measure the effects of KHF signals on the rat tibial nerve in vivo. An incision was made on the left hind limb from the distal dorsal ankle to 1 cm rostral to the ipsilateral hip joint. The muscle overlying the gastrocnemius was cut parallel to the skin incision to expose the gastrocnemius and the sciatic nerve. The connective tissue surrounding the sciatic nerve was dissected from ˜0.5 cm caudal to the spinal cord to the branching point into the tibial, common peroneal, and sural nerves. The common peroneal and sural nerves were transected, as well as the branches of the sciatic nerve innervating the hamstring, leaving only the tibial branch intact. The gastrocnemius was dissected from the tibia. The Achilles tendon was dissected and cut at its distal end, and the tendon was tied to a custom strain gauge-based force transducer using umbilical tape. The tibia was secured at its caudal end by a plastic clamp that was attached to the experimental table.

A tripolar cuff was placed on the proximal sciatic nerve to deliver test pulses to contract the gastrocnemius and a bipolar cuff on the distal sciatic nerve to deliver the KHF waveforms. The tripolar cuff (1 mm inner diameter; X-Wide Contact Cuffs, Microprobes; Gaithersburg, Md.) contained three Pt-Ir 90-10 ribbon contacts (0.5 mm wide) spaced 1 mm apart edge-to-edge; the cuff was 6.5 mm in length total, including 1.5 mm of silicone beyond the outer edge of each outer contact. The bipolar cuff (1 mm inner diameter; X-Wide Contact Cuffs, Microprobes; Gaithersburg, Md.) contained two Pt-Ir 90-10 ribbon contacts (0.5 mm wide) spaced 1 mm apart edge-to-edge; the cuff was 5 mm in length total, including 1.5 mm of silicone on each end. The silicone thickness of both cuffs was 0.875 mm. After implanting the cuffs at the start of each experiment, the impedance was measured between the middle contact and the shorted outer contacts of the tripolar cuff (impedance at 10 kHz: 0.82 to 1.30 kΩ; median=0.92 kΩ) and between the contacts of the bipolar cuff (impedance at 10 kHz: 2.00 to 3.20 kΩ; median=2.70 kΩ). After placement, the two cuffs were spaced ˜0.2 to 0.5 cm edge-to-edge.

Stimulation signals and recorded muscle force were controlled and sampled by a computer and PowerLab/4SP (ADInstruments Inc.; Colorado Springs, Colo.). Custom MATLAB scripts controlled and synchronized all stimulation and recording protocols. The signals from the force transducer were amplified at 10× (ETH-255; CB Sciences Inc.; Dover, N.H.) and were digitized and recorded by the PowerLab unit interfaced via LabChart v7.0 (fₛ=200 samples/s, 50 Hz digital low pass filter; ADInstruments). Voltage signals from the PowerLab unit drove a voltage-to-current stimulus isolator (A-M Systems 2200, Sequim, Wash.) to deliver biphasic symmetric test pulses (0.2 ms/phase) to the tripolar cuff (cathodal phase first to the middle contact and anodal phase first to the shorted outer contacts) via a DC offset removal circuit (100 kΩ resistor in parallel with the stimulus isolator and a 1 μF capacitor in series with the isolator output; based on a previous study). The test pulses had higher amplitudes than required to generate maximal twitches of the gastrocnemius muscle (˜0.7 to 1 mA). A voltage-to-current high power stimulus isolator with 1 MHz bandwidth (A-M Systems 4100) delivered KHF waveforms to the bipolar cuff with the positive output connected to the proximal contact such that “cathodal” or “anodal” stimulation from the computational models matched “cathodal” or “anodal” stimulation from experiments. The KHF signals were generated by a computer-controlled current source (Keithley 6221) that was triggered by MATLAB through a National Instruments VISA connection; the output of the Keithley was passed through a 100Ω resistor and the voltage across this resistor was supplied as input to the A-M Systems 4100 on the 10× input gain setting. A DC offset removal circuit was not included between the KHF signal source and the cuff electrode because an explicit goal of the study was to evaluate the effects of charge imbalances. Rather, prior to every experiment, the A-M Systems 4100 was calibrated such that shunting its inputs produced less than 2 μA DC offset current at the output across a 1 kΩ resistor. In addition, the KHF signal was monitored during the experiments by visualizing the voltage across a 100 SI resistor in series with the bipolar cuff using a battery-powered oscilloscope (Fluke 190-062 ScopeMeter Test Tool; Fluke Corporation; Everett, Wash., USA).

Block threshold (i.e., the minimum current required to produce nerve block) was measured for each waveform-frequency pair using a low-to-high search followed by a binary search. The order of all waveforms to be tested was randomized, and then the order of the four frequencies were randomized for each waveform (20 to 80 kHz, Δ=20 kHz). During each test, a KHF signal was applied at an initial amplitude between 1 to 3.5 mA (charge-balanced waveforms) or between 0.2 to 0.5 mA (charge-imbalanced waveforms). The amplitude was increased if the initial amplitude did not block and this process was repeated until a supra-block amplitude was identified. A standard binary search was conducted by iteratively applying the mean of the largest non-blocking amplitude and the smallest blocking amplitude until a difference between the search bounds of less than 0.2 mA (charge-balanced waveforms) or 0.1 mA (charge-imbalanced waveforms) was observed. Test pulses were applied at 1 Hz, except for the charge-imbalanced waveforms tests at 80 kHz, where 2 Hz was used due to the short duration of those tests (see below). The presence or absence of nerve block was determined visually based on the presence or absence of gastrocnemius contraction in force recordings displayed in real-time in LabChart.

Three strategies were employed to reduce the application of non-zero net charge and therefore reduce the risk of permanent impairment of nerve conduction. Initial KHF amplitudes were set to be markedly lower for charge-imbalanced waveforms, as stated above, and the duration of each delivery of a KHF signal was short: 2 s (80 kHz), 3 s (60 kHz), 4 s (40 kHz), or 5 s (20 kHz) for the charge-imbalanced waveforms and 5 s for all charge-balanced waveforms. Further, for a given waveform, frequency, and amplitude, both polarities (i.e., cathodal and anodal) were evaluated consecutively (with 2 s pause in between) to achieve zero net charge over each pair of tests. A >2 s pause was allowed between amplitudes and >5 s between each waveform and frequency pair. In addition to expediting the experiment, the short duration signals and low initial amplitudes also reduced the possibility of confounding carryover effects, which were not observed in this study. In nerves 1-3, each binary search was terminated after identifying the minimum amplitude that blocked nerve conduction regardless of polarity, taking the block threshold only of the polarity that blocked at a lower threshold. In nerves 4-7, each threshold search was extended to measure block threshold at both polarities consecutively when polarity effects were evident.

Rats were euthanized at the termination of experiments with Euthasol (0.5 ml IP; Virbac; Fort Worth, Tex., USA) and bilateral thoracotomy within 12 hr of the initial urethane dose.

### Example 5

Non-monotonic block thresholds across frequencies are due to amplitude- and frequency-dependent charge imbalance. The block thresholds for a suite of symmetric and asymmetric biphasic kilohertz frequency (KHF) waveforms were quantified (FIG. 1), including charge-balanced and -imbalanced waveforms, using both computational models and in vivo experiments (FIG. 2). A finite element model of the rat tibial nerve coupled to biophysically-realistic models of myelinated axons was implemented. The rat tibial nerve was stimulated in vivo and the resulting gastrocnemius force was recorded.

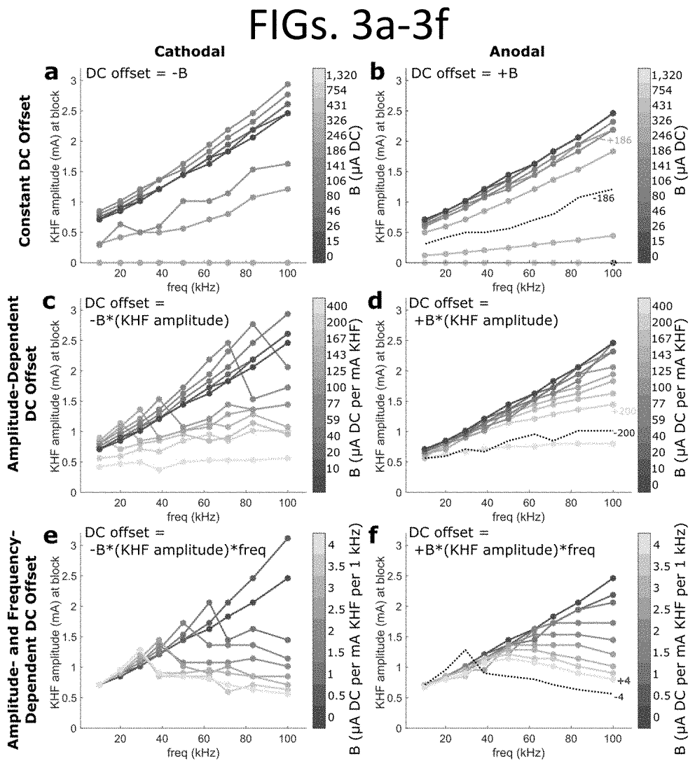

First, block thresholds were investigated using symmetric rectangular waves with various DC offsets (FIGS. 1a-1c). The effects of DC offsets differed with the type (constant, amplitude-dependent, amplitude- and frequency-dependent; FIG. 1c), amount, and polarity of DC. Quantifying the effects of DC offsets on block thresholds in a computational model of 5.7 μm myelinated fibers from 10 to 100 kHz revealed that non-monotonic effects of frequency on block threshold resulted from amplitude- and frequency-dependent charge imbalances (FIG. 3).

Small amounts of constant DC had polarity-dependent effects on block thresholds, but in all cases, block thresholds increased with frequency for a given constant level of DC (FIGS. 3a-3b). Comparing across levels of DC, cathodal DC (i.e., net cathodal current on the proximal contact; FIG. 2) up to −106 μA increased block thresholds for all frequencies (FIG. 3a), while anodal DC up to +246 μA decreased block thresholds for all frequencies (FIG. 3b). Block thresholds dropped abruptly at higher levels of constant cathodal (beyond −141 μA) and anodal (beyond+326 μA) DC, reaching zero for both polarities by ±431 μA; thresholds of zero corresponded to the DC component producing nerve block on its own, irrespective of KHF amplitude or frequency.

Cathodal DC offsets that scaled with KHF amplitude either increased block thresholds at a given frequency when frequencies were low, or decreased thresholds when frequencies were high (FIGS. 3c, −59 to −167 μA per mA KHF). This transition happened at a particular ‘knee’ frequency that was inversely related to the magnitude of DC offset (i.e., parameter “B” in FIG. 3c). Below the knee frequency, the effects of cathodal DC were qualitatively similar to smaller amplitudes of constant cathodal DC (e.g., FIGS. 3a, −15 to −141 μA). Above the knee frequency, the effects were similar to larger amplitudes of constant cathodal DC (e.g., FIG. 3a, −186 μA). Importantly, block thresholds increased monotonically with frequency before and after the knee frequency. Anodal DC offsets (FIG. 3d) that scaled with KHF amplitude decreased block thresholds at any given frequency, similar to constant anodal DC offsets (FIG. 3b). Block thresholds for amplitude-dependent DC did not drop to zero because the DC amplitude was dependent on the KHF amplitude so DC block could not occur at zero. However, by 400 μA DC per mA KHF, the effects of frequency on block thresholds were substantially muted for both polarities.

DC offsets that scaled with both KHF amplitude and frequency (FIGS. 3e-3f) uniquely produced block thresholds that changed non-monotonically with frequency, first increasing and then decreasing as frequency was increased. Cathodal DC offsets that were dependent on both KHF amplitude and frequency exhibited a ‘knee’ frequency (FIG. 3e) similar to those of FIG. 3c, except that thresholds decreased with frequency after the ‘knee’ (FIG. 3e). Anodal DC offsets that were dependent on both KHF amplitude and frequency produced lower block thresholds with greater offset (FIG. 30 similar to effects in FIG. 3d, except that thresholds increased then decreased with frequency at DC offset levels greater than or equal to 1.5 μA DC per mA KHF per 1 kHz for the range of frequencies examined.

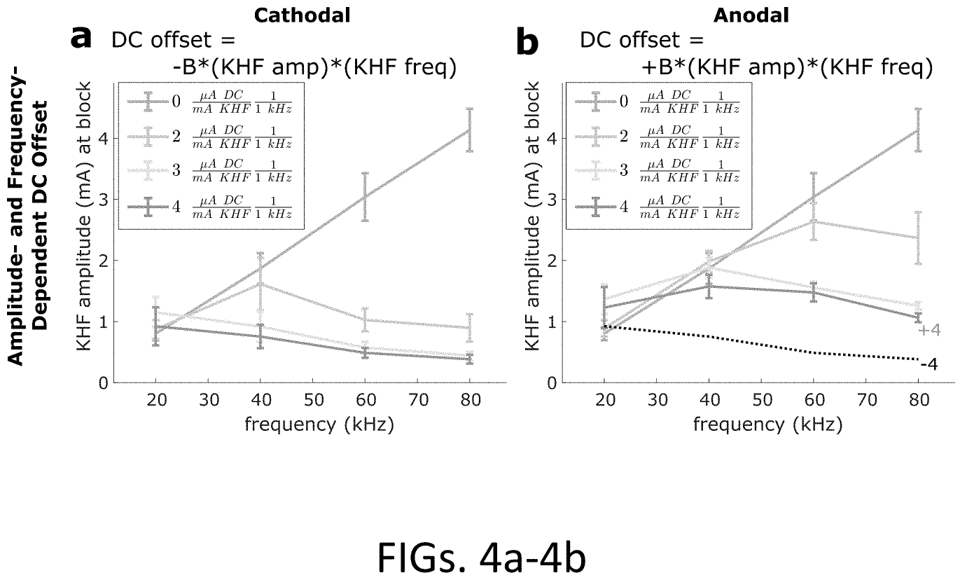

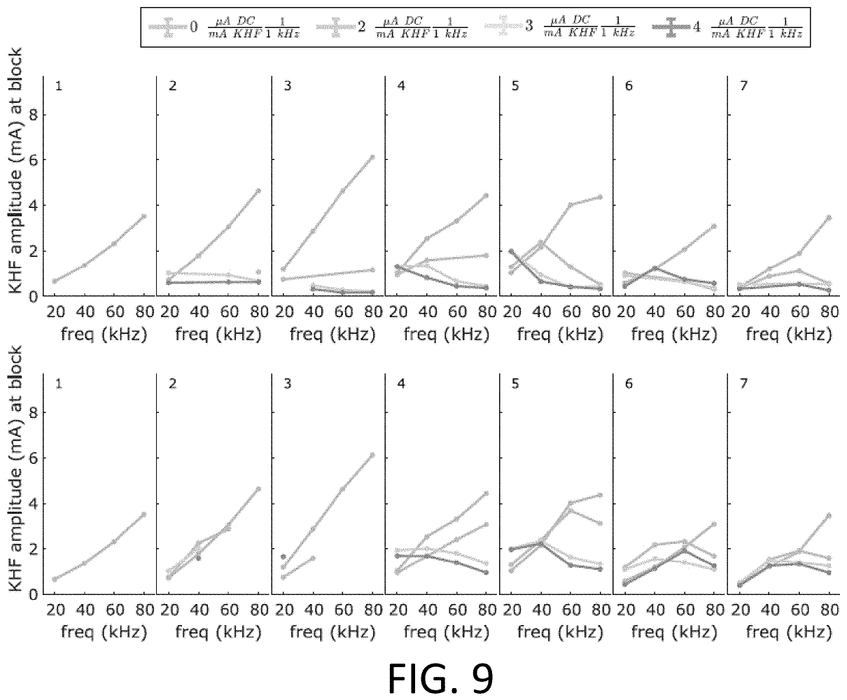

In vivo experiments confirmed the non-monotonic frequency effects of amplitude- and frequency-dependent DC offsets for symmetric waveforms (FIG. 4; FIG. 9). In all rat tibial nerves tested, KHF signals with zero DC offset exhibited block thresholds that increased monotonically with frequency. Conversely, all waveforms with DC offsets that depended on both KHF amplitude and frequency exhibited block thresholds that varied non-monotonically with frequency. Waveforms with greater DC offset magnitude (i.e., parameter “B” in FIG. 4) generally exhibited lower block thresholds at a given frequency and a maximum threshold that occurred at a lower frequency. Equation 3 fits showed a linear relationship between the degree of DC offset (|L|*f) and the natural log of block thresholds (m=2.3; CI=[2.0, 2.6]; adjusted R²=0.69; F(11,123)=27.68; p-value=3e-28), with minor deviations of residuals from normality (Anderson-Darling test p-value: 0.0263).

In computational models and in vivo experiments, cathodal DC offsets of a given level reduced block thresholds more than modal DC offsets of the same level (examples marked in black dashed lines and corresponding labeled colored lines in FIGS. 3b, 3d, 3f and FIG. 4b). Exceptions occurred in computational models when cathodal DC offsets were small enough to increase block thresholds (e.g., below knee frequency), although this phenomenon was not consistent during in vivo experiments (FIG. 4b).

### Example 6

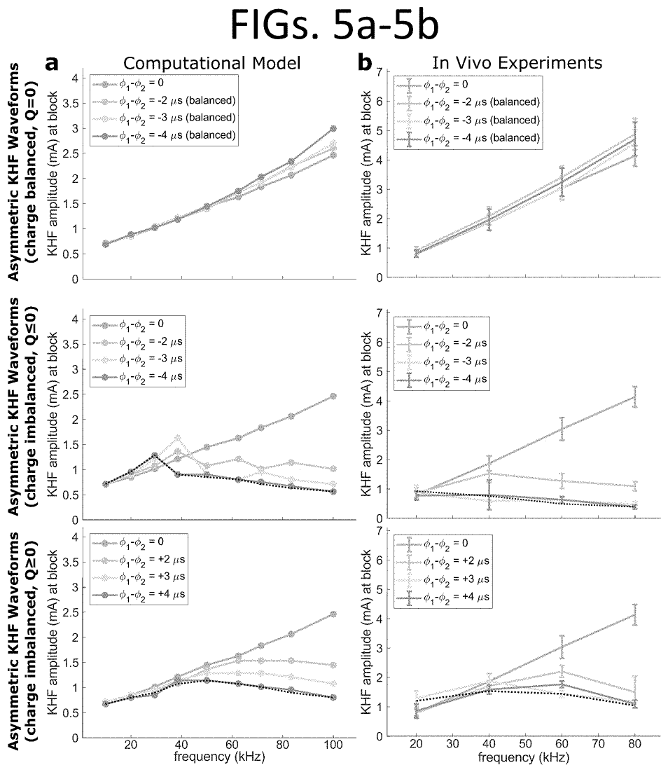

Charge-imbalanced asymmetry but not charge-balanced asymmetry produced non-monotonic threshold-frequency relationships. While the above sections examined charge-balanced and -imbalanced symmetric waveforms, experiments were also conducted to examine the responses to asymmetric waveforms (FIGS. 1d & 1e). In computational models (FIG. 5a) and in vivo experiments (FIG. 5b), block threshold increased monotonically with frequency for charge-balanced asymmetric waveforms, and asymmetry had little to no effect on peak-to-peak KHF amplitude at block threshold, with slight increases in block threshold due to asymmetry at ≥60 kHz in computational models. Conversely, non-monotonic block threshold-frequency relationships were observed with charge-imbalanced asymmetric waveforms. The effects of charge-imbalanced asymmetric waveforms were similar to the effects of symmetric waveforms that had an equivalent level of amplitude- and frequency-dependent DC offset (e.g., FIG. 5 black dashed lines vs. orange lines comparing ±4 μs phase differences vs. ±4 μA DC offset per mA KHF per 1 kHz, data from FIG. 3e, 3f and FIG. 4a, 4b). The trends observed were consistent across computational models and in vivo experiments. Equation 3 fits showed a linear relationship between the degree of net charge imbalance per unit time (|L|*f) and the natural log of block thresholds (m=2.2; CI=[2.0, 2.5]; adjusted R²=0.65; F(11,132)=24.93; p-value=4e-27), with normal residuals (Anderson-Darling test p-value: 0.4413), and this was consistent with effects of DC offset in symmetric waveforms. Therefore, the non-monotonic effects of charge imbalance occurred irrespective of whether the charge imbalance was due to translational DC offsets or an equivalent amount of charge per unit time from unequal phase durations.

### Example 7

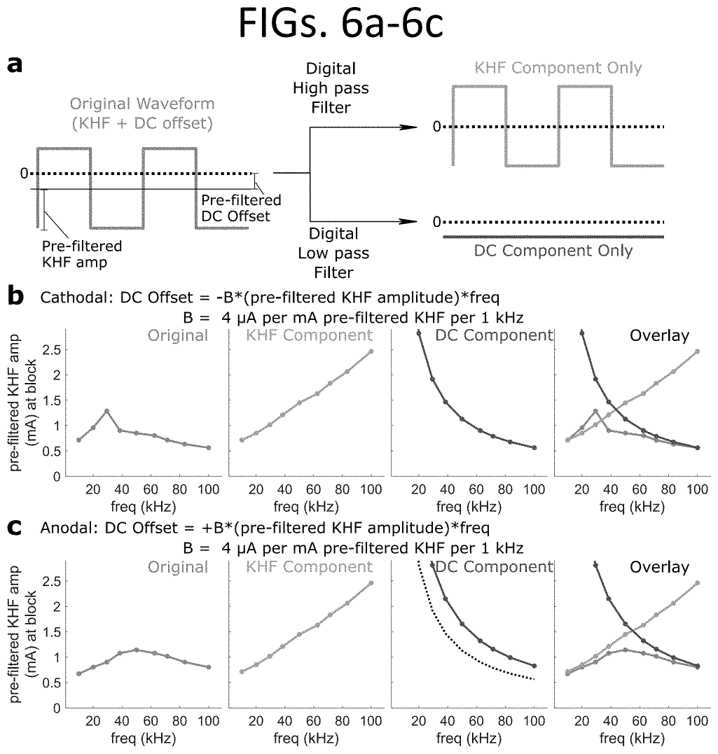

Non-monotonic block thresholds transitioned from charge-balanced KHF thresholds at low frequencies to amplitude- and frequency-dependent DC thresholds at high frequencies. The contributions of the KHF and DC components of the signals to the production of conduction block were quantified for symmetric waveforms with amplitude- and frequency-dependent DC offsets of ±4 μA DC per mA KHF per 1 kHz (FIG. 1c, subpanel c3) in a computational model of a 5.7 μm diameter fiber. To isolate the effects of the KHF and DC components, the waveforms were filtered to preserve either the KHF component only (high pass) or the DC offset component only (low pass) (FIG. 6a), and the block threshold for each component was identified separately.

Non-monotonic changes in block threshold with frequency reflected a transition from a purely KHF block regime at low frequencies, where the DC component of waveforms was small, to a block regime at high frequencies that was solely the result of the DC component as a consequence of the frequency- and amplitude-dependent increase in net DC offsets. The original waveforms resulted in non-monotonic block thresholds with frequency, and the KHF components of the original waveforms had thresholds that increased monotonically with frequency irrespective of the original waveform's DC offset polarity (FIGS. 6b-6c). These results were identical to results for ±4 μA DC per mA KHF per 1 kHz DC offset waveforms (i.e., the original waveforms) and for the 0 μA DC waveforms (i.e., KHF component only) shown in FIGS. 3e-3f The DC offset components of the original waveforms had monotonically decreasing block thresholds regardless of DC offset polarity (FIGS. 6b-6c), reflecting the fact that the DC component of the original waveform had a larger magnitude at higher frequencies due to the DC offset being dependent on the original waveform's KHF amplitude and frequency (FIG. 1c, subpanel c3). Therefore, at higher frequencies, the DC offset components extracted from the original waveform required a smaller pre-filtered KHF amplitude to reach DC block threshold. Block thresholds for the original waveforms approached the thresholds for the KHF-only components at lower frequencies and approached the thresholds for the DC offset components at higher frequencies, irrespective of DC offset polarity (FIGS. 6b-6c, Overlay), indicating that a transition from KHF to DC block underlays the non-monotonic threshold-frequency relationships of the original waveforms.

Cathodal DC components alone had lower block thresholds than anodal DC components alone (FIG. 6c, purple vs. black dotted lines), consistent with differences observed for symmetric waveforms at high frequencies with anodal versus cathodal DC offsets (FIG. 3f, −4 vs. +4 μA DC per mA KHF per 1 kHz lines). This polarity difference was due to anodal DC at the proximal contact augmenting incoming action potentials, as a result of sodium channel de-inactivation, allowing them to propagate through the distal cathode that otherwise could block action potentials when cathodal DC was at the proximal contact.

The analysis further revealed that polarity-dependent differences in non-monotonic threshold-frequency relationships were due to polarity-dependent interactions between KHF and DC components during the transition from KHF to DC block regimes. For waveforms with anodal DC offsets, the transition was relatively smooth across frequencies, and block thresholds were always less than or equal to the KHF or DC components' block thresholds. This result indicated a synergy between KHF and anodal DC (i.e., anodal DC at the proximal contact with cathodal DC at the distal contact) at all frequencies. In contrast, for waveforms with cathodal DC offsets, the transition was marked by an abrupt drop in thresholds after the ‘knee’ frequency (FIG. 6b, orange line, 29.4 vs. 38.5 kHz). Further, block thresholds leading up to this ‘knee’ frequency were greater than the KHF components' block thresholds, but always less than or equal to the DC component's block thresholds. This result indicated a reduced ability of KHF to block in the presence of cathodal DC offsets (i.e., before the ‘knee’) despite KHF always assisting the production of DC block (i.e., after the ‘knee’).

### Example 8

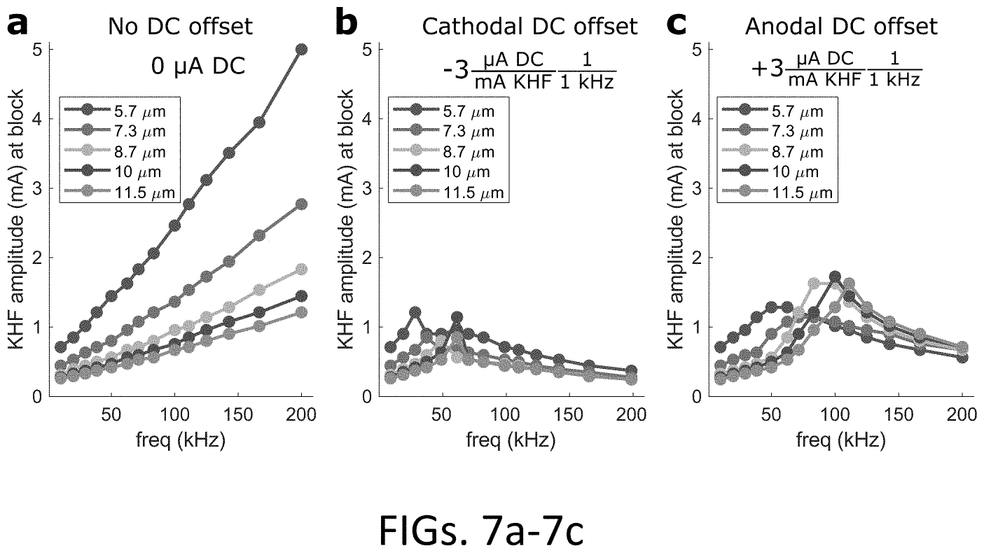

Frequency-dependent charge imbalance blocked some smaller fibers at lower thresholds than larger fibers. Using the computational models described herein, the frequency-dependent effects on block thresholds of symmetric rectangular waveforms were compared with different DC offsets across fiber diameters (5.7, 7.3, 8.7, 10.0, 11.5 λm), extending the upper range of frequencies to observe frequency effects fully (111.1, 125, 142.6, 166.7, and 200 kHz). Block thresholds of KHF waveforms with no DC offset increased monotonically with frequency for all fiber diameters (FIG. 7a), while KHF waveforms with frequency- and amplitude-dependent charge imbalances produced non-monotonic threshold-frequency relationships for all fiber diameters (FIGS. 7b-7c). Further, block thresholds at any given frequency were inversely related to fiber diameter when no DC offsets were present (FIG. 7a), while non-monotonic frequency effects for both cathodal and anodal DC offsets resulted in instances where the order of block was reversed (FIGS. 7b-7c), such that smaller diameter fibers had lower block thresholds than larger diameter fibers. For cathodal DC offsets, such reversals occurred at specific frequencies and for specific fiber diameters (e.g., FIG. 7b, 10.0 μm vs. 5.7 μm at 62.5 kHz). For anodal DC offsets, reversals occurred starting at 71.4 kHz and were maintained across higher frequencies (FIG. 7c), resulting in reversal of block thresholds across all fiber diameters by 111.1 kHz.

### Example 9

Interactions between KHF signal and DC offset modulated excitation and block regions. These results demonstrated that DC modulation of KHF block thresholds created non-monotonic relationships between block threshold and frequency when the DC offset was amplitude- and frequency-dependent. However, block threshold alone does not reflect the range of effects of KHF signals across amplitudes. Other responses, including transmission, excitation, and the extent of block across amplitudes (i.e., the block window) are highly relevant for in vivo application of block. Therefore, the responses to KHF rectangular waveforms mixed with DC in computational models of 5.7 μm diameter myelinated fibers were further characterized by analyzing the number of action potentials detected across amplitudes and frequencies of the KHF signals.

Quantifying model responses across frequencies and amplitudes revealed that DC offsets caused gradual migration of KHF transmission, excitation, and block regions in ways that depended on the amount, polarity, and type of DC offsets. At low KHF amplitudes, waveforms with no DC offset (FIG. 1a) had no effect on action potentials produced by test pulses, i.e., transmission occurred (FIG. 8, 0 μA DC, gray dots). As the KHF amplitude was increased for a given frequency, the response progressed through tonic excitation by the KHF signal, conduction block, and then re-excitation (i.e., excitation by the KHF signal at amplitudes above the block threshold). The range of amplitudes and frequencies that blocked axonal conduction formed a single contiguous region. Excitation, block, and re-excitation thresholds increased with frequency.

Anodal DC offsets of all three types (FIG. 1c) decreased the KHF amplitudes needed for KHF excitation, increased the KHF amplitudes needed for KHF re-excitation, and produced an additional transmission ‘region’ at KHF amplitudes just below block threshold (FIG. 8, +141 μA DC, +77 μA DC per mA KHF, +3 μA DC per mA KHF per 1 kHz). Cathodal DC offsets of all three types had the opposite effect on KHF excitation and KHF re-excitation, and further produced an additional block ‘region’ and an additional excitation ‘region’ at KHF amplitudes below KHF excitation (FIG. 8, −141 μA DC, −77 μA DC per mA KHF, −3 μA DC per mA KHF per 1 kHz). The additional transmission and block regions introduced by anodal and cathodal DC offsets, respectively, occurred at similar KHF amplitudes and frequencies, such that a given KHF signal could block action potentials coming from one direction but transmit action potentials coming from the other direction. The ‘knee’ frequency for −3 μA DC per mA KHF per 1 kHz coincided with an abrupt transition from one block region to another. Together with the results from FIG. 6, which showed that the ‘knee’ represents a transition from a KHF to a DC block regime, these analyses indicate that the second block region introduced by cathodal DC offsets is a DC block region. Only the amplitude- and frequency-dependent DC offsets produced non-monotonic transmission, excitation, and block boundaries. These analyses demonstrate the complex effects that DC offset can have on transmission, block, and excitation in response to KHF signals.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

1. A method for selective nerve fiber conduction block using a neuromodulation device, the method comprising:
applying a hybrid waveform comprising a kilohertz frequency (KHF) component and a direct current (DC) component to a target nerve fiber or set of nerve fibers;
wherein the hybrid waveform achieves conduction block in the target nerve fiber or set of nerve fibers.

2. The method of claim 1, wherein the KHF component comprises a biphasic alternating current waveform.

3. The method of claim 1, wherein the KHF component comprises a waveform with more than two phases.

4. The method of claim 1, wherein the DC component comprises a DC offset superimposed on the KHF component.

5. The method of claim 1, wherein the DC component comprises unequal phase durations, unequal phase amplitudes, and/or unequal phase shapes in the KHF component.

6. The method of claim 1, wherein the hybrid waveform is repeated at a frequency of about 1 kHz to about 200 kHz.

7. The method of claim 1, wherein the hybrid waveform comprises a net charge imbalance per unit time.

8. The method of claim 7, wherein the net charge imbalance is obtained by:
(a) adjusting the amplitude of the DC offset superimposed on the KHF component;
(b) adjusting the magnitude of the difference in the phase durations of the KHF component;
(c) adjusting the magnitude of the difference in the amplitudes of the phases of the KHF component; and/or
(d) adjusting the shapes of the phases of the KHF component;
and any combinations of (a)-(d).

9. The method of claim 1, wherein the method further comprises adjusting polarity of the DC component.

10. The method of claim 1, wherein the hybrid waveform blocks conduction in the target nerve fiber or set of nerve fibers but does not block conduction in a reference nerve fiber or set of nerve fibers.

11. The method of claim 10, wherein the target nerve fiber or set of nerve fibers comprises a diameter(s) that is smaller than the reference nerve fiber.

12. The method of claim 11, wherein the reference nerve fiber comprises a diameter that is from about 0.5 μm to about 20.0 μm; and/or wherein the target nerve fiber or set of nerve fibers comprises a diameter(s) from about 0.2 μm to about 19.5 μm.

13-18. (canceled)

19. The method of claim 10, wherein the target nerve fiber or set of nerve fibers comprises a diameter(s) that is larger than the reference nerve fiber.

20. The method of claim 19, wherein the reference nerve fiber comprises a diameter that is from about 0.2 μm to about 19.5 μm; and/or wherein the target nerve fiber or set of nerve fibers comprises a diameter(s) from about 0.5 μm to about 20.0 μm.

21. (canceled)

22. The method of claim 1, wherein the hybrid waveform comprises a repetition frequency of about 1 kHz to about 200 kHz.

23-32. (canceled)

33. A system for selective nerve fiber conduction block, the system comprising:
an electrode with one or more metal contacts sized and configured for implantation in proximity to neural tissue; and
a pulse generator coupled to the electrode, the pulse generator including a power source comprising a battery and a microprocessor coupled to the battery;
wherein the pulse generator is capable of applying to the electrode a hybrid waveform capable of achieving selective conduction block in a target nerve fiber or set of nerve fibers.

34-35. (canceled)

36. The system of claim 33, wherein the hybrid waveform blocks conduction in the target nerve fiber or set of nerve fibers but does not block conduction in a reference nerve fiber or set of nerve fibers.

37. A method for obtaining selective nerve fiber conduction block using the system of claim 33 comprising programming the pulse generator to output the hybrid waveform, wherein the hybrid waveform blocks neural conduction when delivered by the pulse generator.

38. A method for obtaining unidirectional nerve fiber conduction block using a neuromodulation device, the method comprising:
applying a hybrid waveform comprising a kilohertz frequency (KHF) component and a direct current (DC) component to a target nerve fiber or set of nerve fibers;
wherein the hybrid waveform achieves a conduction block in the target nerve fiber or set of nerve fibers in a unidirectional manner.

39-43. (canceled)

44. The method of claim 38, wherein the hybrid waveform comprises a charge imbalance obtained by:
(a) adjusting unequally the amplitudes of the phases of the KHF component;
(b) adjusting the magnitude of the difference in the phase duration of the KHF component;
(c) adjusting the amplitude of the DC offset superimposed on the KHF component; and/or
(d) adjusting the shapes of the phases of the KHF components;
and any combinations of (a)-(d).

45-53. (canceled)
